(19) United States
(12) Patent Application Publication
(10) Pub. No.: US 2010/0113276 A1
(43) Pub. Date: May 6, 2010

(54) USE OF N2-PHENYLAMIDINES AS HERBICIDES

(75) Inventors: Birgit Kuhn, Kelkheim (DE); Harald Jakobi, Frankfurt (DE); Thomas Mueller, Frankfurt (DE); Joerg Tiebes, Frankfurt (DE); Heinz Kehne, Hofheim (DE); Dirk Schmutzler, Hattersheim (DE); Martin Hills, Idstein (DE); Christopher Rosinger, Hofheim (DE); Klaus Kunz, Duesseldorf (DE); Mark Wilheim Drewes, Langenfeld (DE); Dieter Feucht, Eschborn (DE); Thomas Seitz, Langenfeld (DE); Benoit Hartmann, Sainte Foy-les-Lyon (FR); Ralf Dunkel, Leichlingen (DE); Joerg Nico Greul, Leichlingen (DE); Oliver Guth, Leverkusen (DE); Kerstein Ilg, Koeln (DE); Darren James Mansfeld, Kuerten (DE); Wahed Ahmed Moradi, Monheim (DE); Peter Dahmen, Neuss (DE); Ulrike Wachendorf-Neumann, Neuwied (DE); Arnd Voerste, Koeln (DE); Dale Robert Mitchell, Essex (GB); Jean-Pierre Andre Vors, Sainte Foy-les-Lyon (FR); Dale Robert Mitchell, Essex (GB); Tom Hough, Cambridge (GB)

Correspondence Address: Baker Donelson Bearman, Caldwell & Berkowitz, PC, 555 Eleventh Street, NW, Sixth Floor, Washington, DC, 20004, US

(73) Assignee: BAYER CROPSCIENCE AG, Monheim (DE)

(21) Appl. No.: 12/530,781

(22) PCT Filed: Mar. 4, 2008
(86) PCT No.: PCT/EP2008/001683

§ 371 Date: Jan. 20, 2010

(30) Foreign Application Priority Data
Mar. 12, 2007 (EP) 07005003.4
Mar. 12, 2007 (EP) 07005004.2
Mar. 12, 2007 (EP) 07005006.7
Jun. 27, 2007 (DE) 10 2007 029 603.9

(51) Int. Cl. A01N 43/54; C07C 257/10; A01N 35/10; C07D 213/62; A01N 43/40; C07D 213/00; C07D 317/10; A01N 43/28; C07D 285/02; A01N 43/82; C07D 277/00; A01N 43/78; C07D 231/02; A01N 43/56; C07D 285/08; A01N 43/836; C07D 239/02; C07D 285/12; A01N 43/824; A01P 13/00

(52) U.S. Cl. 504/242; 564/245; 504/343; 546/300; 504/256; 546/332; 504/244; 549/449; 504/295; 548/125; 504/261; 548/146; 504/266; 548/366.1; 504/282; 548/129; 504/262; 544/298; 548/136; 504/263

(57) ABSTRACT

The use of N²-phenylamidines of formula (I) as herbicides is described.

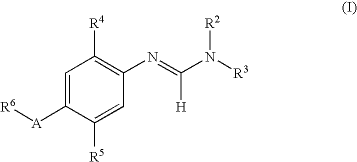

In this formula (I), R², R³, R⁴, R⁵ and R⁶ are different radicals and A is a bond or various 1- or polyatomic bridging elements.

The invention relates to the technical field of herbicides, in particular to that of herbicides for selectively controlling broad-leaved weeds and weed grasses in crops of useful plants.

It is already known from various publications that certain phenylamidines have fungicidal properties. For example, EP 1 150 944 B1 describes fungicidally active N²-phenylamidines which carry on the phenyl ring—inter alia—a carbocyclic or heterocyclic radical bonded directly or via a mono- or polyatomic group.

The herbicidal effect of such compounds has hitherto not been described.

It was an object of the present invention to provide herbicidally effective compounds.

It has now been found that N²-phenylamidines of formula (I), or salts thereof, have excellent herbicidal properties.

The present invention provides the use of compounds of formula (I), or salts thereof, as herbicides

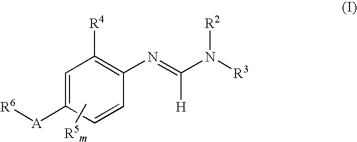

in which
R² and R³, independently of one another, are each (C₁-C₆)-alkyl, (C₃-C₆)-cycloalkyl, (C₂-C₆)-alkenyl, (C₂-C₆)-alkynyl, halo-(C₁-C₆)-alkyl, halo-(C₂-C₆)-alkenyl, halo-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl, preferably independently of one another in each case (C₁-C₆)-alkyl, (C₂-C₆)-alkenyl, (C₂-C₆)-alkynyl, halo-(C₁-C₆)-alkyl, halo-(C₂-C₆)-alkenyl, halo-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl,
or R² and R³ are together (CH₂)₄ or (CH₂)₅,
or
R² and R³ together with the nitrogen atom to which they are bonded, form a 5- or 6-membered saturated, partially saturated, unsaturated or aromatic ring which comprises k heteroatoms from the group consisting of oxygen, nitrogen and sulfur and which is substituted by p radicals from the group consisting of halogen, methyl, ethyl, methoxy, ethoxy, trifluoromethyl, nitro, cyano and hydroxy,
R⁴ and R⁵ independently of one another are each (C₁-C₆)-alkyl, (C₂-C₆)-alkenyl, (C₂-C₆)-alkynyl, (C₃-C₆)-cycloalkyl, halogen, cyano, hydroxy, mercapto, acyl, OR^{a}, SR^{a}, Si(R^{a})₃ halo-(C₁-C₆)-alkyl, (C₁-C₄)-alkoxy-(C₁-C₆)-alkyl or heterocyclyl bonded to phenyl via a carbon atom,
R^{a} is (C₁-C₈)-alkyl,
m is 1, 2 or 3,
R⁶ is in each case carbocyclyl or heterocyclyl substituted by n radicals from the group consisting of halogen, cyano, phenoxy, (C₁-C₈)-alkylcarbonyl, (C₁-C₈)-alkoxycarbonyl, (C₁-C₈)-alkyl, (C₁-C₈)-alkoxy, (C₂-C₈)-alkenyl, (C₂-C₈)-alkynyl and 1,3-dioxolan-2-yl,
   where the specified radicals (C₁-C₈)-alkyl, (C₁-C₈)-alkoxy, (C₂-C₈)-alkenyl and (C₂-C₈)-alkynyl are substituted by n radicals from the group consisting of (C₁-C₈)-alkoxy, hydroxy and halogen and where
1,3-dioxolan-2-yl is substituted by n radicals (C₁-C₈)-alkyl,
A is a bond or a divalent group —O—, —S(O)ₙ—, —NR⁹, —CR⁷═CR⁷—, —C≡C—, -A¹-, -A¹-A¹-, -A²-, -A³-, -A¹O—, -A¹S(O)ₙ—, —OA²-, —NR⁹-A²-,
   —OA²-A¹-, —OA²-CR⁷═CR⁸—, —S(O)ₙ-A¹-, —(CH₂)₂—ON═CR⁸—, —X-A²-NH—, —C(R⁸)═NO—(C₁-C₆)-alkyl or —O(A¹)ₖO—,
A¹ is in each case —CHR⁷—,
A² is in each case —C(═X)—,
A³ is —CR⁸═NO—,
X is in each case independently of the others oxygen or sulfur,
R⁷ is—in each case independently of other radicals R⁷-hydrogen, halogen, cyano, (C₁-C₆)-alkyl, (C₃-C₆)-cycloalkyl, phenyl, halogen, cyano, hydroxy, mercapto, halo-(C₁-C₆)-alkyl or (C₁-C₄)-alkoxy-(C₁-C₆)-alkyl,
R⁸ is—in each case independently of other radicals R⁸-hydrogen, (C₁-C₆)-alkyl, (C₂-C₆)-alkenyl, (C₂-C₆)-alkynyl, (C₁-C₆)-alkoxy, (C₁-C₆)-alkylthio, (C₃-C₆)-cycloalkyl, phenyl, halogen, cyano, hydroxy, mercapto, halo-(C₁-C₆)-alkyl, (C₁-C₄)-alkoxy-(C₁-C₆)-alkyl, carbocyclyl or heterocyclyl,
R⁹ is—in each case independently of other radicals R⁹-hydrogen, (C₁-C₆)-alkyl, carbocyclyl or heterocyclyl,
k is—in each case independently of other variables k −1, 2 or 3,
n is—in each case independently of other variables n −0, 1 or 2, and
p is 0, 1, 2 or 3.

The linkage of A with R⁶ and the phenyl ring should be understood as meaning that R⁶ is bonded on the right-hand side and the phenyl ring is bonded on the left-hand side of A.

The compounds of formula (I) can also be present in salt form, for example as hydrochloride or in the form of other acid adducts. These salts are likewise suitable as herbicides and are intended to be encompassed by formula (I). Preference is given to hydrochlorides, hydrobromides, trifluoroacetates, acetates and trifluoro-methanesulfonates.

In formula (I) and all of the formulae below, alkyl radicals having more than two carbon atoms may be straight-chain or branched. Alkyl radicals are, for example, methyl, ethyl, n- or isopropyl, n-, iso-, t- or 2-butyl, pentyl, hexyl, such as n-hexyl, isohexyl and 1,3-dimethylbutyl. Halogen is fluorine, chlorine, bromine or iodine.

If a group is substituted by radicals more than once, then this is to be understood as meaning that this group is substituted by one or more identical or different of the specified radicals.

Heterocyclyl is a saturated, unsaturated or heteroaromatic cyclic radical; it contains one or more heteroatoms in the ring, preferably from the group consisting of N, O and S; preferably, it is an aliphatic heterocyclyl radical having 3 to 7 ring atoms or a heteroaromatic radical having 5 or 6 ring atoms and comprises 1, 2 or 3 heteroatoms.

The heterocyclic radical can be, for example, a heteroaromatic radical or ring (heteroaryl), such as, for example, a mono-, bi- or polycyclic aromatic system in which at least 1 ring comprises one or more heteroatoms, for example pyridyl, pyrimidinyl, pyridazinyl, pyrazinyl, triazinyl, thienyl, thiazolyl, oxazolyl, furyl, pyrrolyl, pyrazolyl and imidazolyl, or is a partially or completely hydrogenated radical such as oxiranyl, pyrrolidyl, piperidyl, piperazinyl, dioxolanyl, morpholinyl, tetrahydrofuryl. Suitable substituents for a substituted heterocyclic radical are the substituents given below, additionally also oxo. The oxo group can also occur on the hetero ring atoms, which can exist in various oxidation states, e.g. in the case of N and S.

Carbocyclyl is a saturated, unsaturated or aromatic cyclic radical which comprises exclusively carbon atoms in the ring; for example cycloalkyl, cycloalkenyl, phenyl and naphthyl. Suitable substituents for carbocyclyl are the substituents specified below, additionally also oxo. The oxo group can also occur on the hetero ring atoms, which can exist in various oxidation states, e.g. in the case of N and S.

Cycloalkyl is a carbocyclic, saturated ring system with three to nine carbon atoms, e.g. cyclopropyl, cyclopentyl or cyclohexyl.

If the term acyl radical is used in this description, this means the radical of an organic acid which is produced formally by eliminating an OH group from the organic acid, e.g. the radical of a carboxylic acid and radicals of acids derived therefrom such as thiocarboxylic acid, optionally N-substituted iminocarboxylic acids or the radicals of carbonic acid monoesters, optionally N-substituted carbamic acids, sulfonic acids, sulfinic acids, phosphonic acids, phosphinic acids.

An acyl radical is preferably formyl or acyl from the group consisting of CO—R^{z}, CS—R^{z}, CO—OR^{z}, CS—OR^{z}, CS—SR^{z}, SOR^{z} or SO₂R^{z}, where R^{z} is in each case a C₁-C₁₀-hydrocarbon radical such as C₁-C₁₀-alkyl or phenyl, which is unsubstituted or substituted, e.g. by one or more substituents from the group consisting of halogen, such as F, Cl, Br, I, alkoxy, haloalkoxy, hydroxy, amino, nitro, cyano or alkylthio, or R^{z} is aminocarbonyl or aminosulfonyl, where the two last-mentioned radicals are unsubstituted, N-monosubstituted or N,N-disubstituted, e.g. by substituents from the group consisting of alkyl or aryl.

Acyl is, for example, formyl, haloalkylcarbonyl, alkylcarbonyl such as (C₁-C₄)-alkylcarbonyl, phenylcarbonyl, where the phenyl ring may be substituted, or alkyloxycarbonyl, such as (C₁-C₄)-alkyloxycarbonyl, phenyloxycarbonyl, benzyl-oxycarbonyl, alkylsulfonyl, such as (C₁-C₄)-alkylsulfonyl, alkylsulfinyl, such as C₁-C₄-(alkylsulfinyl), N-alkyl-1-iminoalkyl, such as N—(C₁-C₄)-1-imino-(C₁-C₄)-alkyl and other radicals of organic acids.

The compounds of formula (I) and salts thereof can be present as stereoisomers depending on the type and linkage of the substituents. If, for example, one or more asymmetric carbon atoms are present, then enantiomers and diastereomers can arise. Stereoisomers can be obtained from mixtures produced during the preparation by customary separation methods, for example by chromatographic separation methods. Stereoisomers can likewise be selectively prepared by using stereoselective reactions using optically active starting materials and/or auxiliaries. The invention also relates to all stereoisomers and mixtures thereof which are encompassed by formula (I) but not specifically defined. In particular, it relates to the E/Z isomers, both their mixture and the individual isomers.

Preference is given to compounds of formula (I), in which
R² and R³ independently of one another, are in each case (C₁-C₆)-alkyl, cyclopropyl, (C₂-C₆)-alkenyl, (C₂-C₆)-alkynyl, halo-(C₁-C₆)-alkyl, halo-(C₂-C₆)-alkenyl, halo-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl, or preferably independently of one another are in each case (C₁-C₆)-alkenyl, (C₂-C₆)-alkenyl, (C₂-C₆)-alkynyl, halo-(C₁-C₆)-alkyl, halo-(C₂-C₆)-alkenyl, halo-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl, or are together (CH₂)₄ or (CH₂)₅,
R⁴ is (C₁-C₆)-alkyl, halo-(C₁-C₆)-alkyl or (C₁-C₄)-alkoxy-(C₁-C₆)-alkyl,
R⁵ is halogen, (C₁-C₆)-alkyl, halo-(C₁-C₆)-alkyl or (C₁-C₄)-alkoxy-(C₁-C₆)-alkyl,
A is a bond, —O—, —S—, —CH₂CH₂—, —CH₂—, —OCH₂—, —CH═CH—, —C≡C—, —NH—CO—, —N(CH₃)—, NH— or —O—CO—NH—,
R⁶ is phenyl or naphthyl substituted by n radicals from the group consisting of halogen, cyano, phenoxy, (C₁-C₄)-alkylcarbonyl, (C₁-C₄)-alkyl, (C₁-C₆)-alkoxy, (C₂-C₆)-alkenyl, (C₂-C₆)-alkynyl and 1,3-dioxolan-2-yl, where the specified radicals (C₁-C₆)-alkyl, (C₁-C₆)-alkoxy, (C₂-C₆)-alkenyl and (C₂-C₆)-alkynyl are substituted by n radicals from the group consisting of (C₁-C₄)-alkoxy, hydroxy and halogen
   and where 1,3-dioxolan-2-yl is substituted by n radicals (C₁-C₅)-alkyl, or R⁶ is heterocyclyl substituted by n radicals from the group consisting of halogen, (C₁-C₆)-alkyl, halo-(C₁-C₄)-alkoxy and halo-(C₁-C₄)-alkyl.
m is 1 and
n is—in each case independently of other variables n −0, 1 or 2.

Particular preference is given to compounds of formula (I), in which
R² is methyl,
R³ is methyl, ethyl, cyclopropyl or isopropyl, or,
   R² and R³ are together (CH₂)₄ or (CH₂)₅,
R⁴ is methyl,
R⁵ is methyl or chlorine,
A is a bond, —O—, —S—, —CH₂—CH₂—, —CH₂—, —OCH₂— or —CH═CH—, in particular a bond or —O—,
R⁶ is phenyl or naphthyl substituted by n radicals from the group consisting of halogen, cyano, phenoxy, (C₁-C₄)-alkylcarbonyl, (C₁-C₄)-alkyl, (C₁-C₆)-alkoxy, (C₂-C₆)-alkenyl, (C₂-C₆)-alkynyl and 1,3-dioxolan-2-yl, where the specified radicals (C₁-C₆)-alkyl, (C₁-C₆)-alkoxy, (C₂-C₆)-alkenyl and (C₂-C₆)-alkynyl are substituted by n radicals from the group consisting of (C₁-C₄)-alkoxy, hydroxy and halogen and where
   1,3-dioxolan-2-yl is substituted by n radicals (C₁-C₈)-alkyl, or R⁶ is pyridinyl, thiadiazolyl or thiazolyl substituted by n radicals from the group consisting of halogen, (C₁-C₆)-alkyl, halo-(C₁-C₄)-alkoxy and halo-(C₁-C₄)-alkyl,
m is 1, and
n is—in each case independently of other variables n −0, 1 or 2.

The compounds of formula (I) are known from EP 1 150 944 B1 and are accessible by the preparation methods described therein.

The compounds of formula (I) have excellent herbicidal effectiveness against a broad spectrum of economically important mono- and dicotyledonous harmful plants. Perennial broad-leaved weeds which are difficult to control and which sprout from rhizomes, root stocks or other permanent organs, are readily attacked by the active ingredients. In this connection, it is generally unimportant whether the substances are applied in the presowing, preemergence or postemergence method. Specifically, by way of example mention may be made of a number of representatives of the mono- and dicotyledonous broad-leaved weed flora which can be controlled by the compounds of formula (I) without any intention of restriction to certain types as a result of the naming. On the side of the monocotyledonous broad-leaved weed species are, for example, Avena, Lolium, Alopecurus, Phalaris, Echinochloa, Digitaria, Setaria and Cyperus species from the annual group and on the side of the perennial species Agropyron, Cynodon, Imperata and Sorghum and also perennial Cyperus species are readily attacked.

In the case of dicotyledonous broad-leaved weed species, the activity spectrum extends to species such as, for example, Galium, Viola, Veronica, Lamium, Stellaria, Amaranthus, Sinapis, Ipomoea, Sida, Matricaria and Abutilon on the annual side, and also Convolvulus, Cirsium, Rumex and Artemisia in the case of the perennial broad-leaved weeds. Under specific crop conditions, harmful plants that occur in rice, such as, for example, Echinochloa, Sagittaria, Alisma, Eleocharis, Scirpus and Cyperus, are likewise controlled in an excellent manner by the compounds of formula (I). If the compounds of formula (I) are applied to the soil surface prior to germination, then either the emergence of the broad-leaved weed seedlings is completely prevented or the broad-leaved weeds grow up to the seed leaf stage, but then stop growing and finally die off after the course of three to four weeks. In the case of application of the active ingredients to the green parts of the plant in the postemergence method, a drastic stop in growth likewise occurs very rapidly following treatment and the broad-leaved weed plants remain in the growth stage present at the time of application or die off altogether after a certain time, meaning that in this way a broad-leaved weed competition harmful for the crop plants is eliminated very early on and in a lasting manner. In particular, the compounds of formula (I) exhibit an excellent effect against Apera spica venti, Chenopodium album, Lamium purpureum, Polygonum convulvulus, Stellaria media, Veronica hederifolia, Veronica persica, Viola tricolor and also against species of Amaranthus, Galium and Kochia.

Although the compounds of formula (I) have excellent herbicidal activity toward mono- and dicotyledonous broad-leaved weeds, crop plants of economically important crops such as, for example, wheat, barley, rye, rice, corn, sugarbeet, cotton and soybeans, are damaged only negligibly, if at all. In particular, they have excellent compatibility in corn, rice, cereals and soybeans. These compounds are therefore very readily suitable for selectively controlling undesired plant growth in agricultural useful plantations or in ornamental plantations.

On account of their herbicidal properties, these compounds can also be used for controlling harmful plants in crops of known or still developing genetically modified plants. The transgenic plants are usually characterized by particularly advantageous properties, for example by resistances to certain pesticides, primarily certain herbicides, resistances to plant diseases or pathogens of plant diseases such as certain insects or microorganisms such as fungi, bacteria or viruses. Other particular properties relate, for example, to the harvest material with regard to amount, quality, storability, composition and special ingredients. For example, transgenic plants with increased starch content or modified quality of the starch or those with a different fatty acid composition of the harvest material are known.

Preferably, the application of the compounds of formula (I) or salts thereof is in economically important transgenic crops of useful plants and ornamental plants, e.g. of cereals such as wheat, barley, rye, oats, millet, rice, manioc and corn and also in crops of sugarbeet, cotton, soybeans, rape, potatoes, tomatoes, peas and other vegetable varieties. The compounds of formula (I) can preferably be used as herbicides in useful plant crops which are resistant to the phytotoxic effects of the herbicides and/or have been rendered resistant by means of genetic engineering, in particular soybeans and corn.

Conventional methods for producing new plants which have modified properties compared to existing plants consist, for example, in classical cultivation methods and the production of mutants. Alternatively, new plants with modified properties can be produced using genetic engineering methods (see e.g. EP-A-0221044, EP-A-0131624). For example, in several cases the following have been described:
genetic modifications of crop plants for the purpose of modifying the starch synthesized in the plants (e.g. WO 92/11376, WO 92/14827, WO 91/19806),
transgenic crop plants which are resistant to certain herbicides of the glufosinate type (e.g. EP-A 0 242 236, EP-A 0 242 246) or glyphosate type (WO 92/00377) or the sulfonylurea type (EP-A-0257993, U.S. Pat. No. 5,013,659),
transgenic crop plants, for example cotton, with the ability to produce Bacillus thuringiensis toxins (Bt toxins) which make the plants resistant to certain pests (EP-A 0 142 924, EP-A 0 193 259).
transgenic crop plants with a modified fatty acid composition (WO 91/13972).

Numerous molecular biological techniques with which new transgenic plants with modified properties can be produced are known in principle, see e.g. Sambrook et al., 1989, Molecular Cloning, A Laboratory Manual, 2nd edition, Cold Spring Harbor Laboratory Press, Cold Spring Harbor, N.Y.; or Winnacker “Gene and Klone [Genes and Clones]”, VCH Weinheim 2nd edition, 1996 or Christou, “Trends in Plant Science” 1 (1996) 423-431). For genetic manipulations of this type, nucleic acid molecules can be introduced into plasmids which permit a mutagenesis or a sequence modification through recombination of DNA sequences. With the help of the aforementioned standard methods it is possible, for example, to undertake base exchange, remove part sequences or add natural or synthetic sequences. To join the DNA fragments with one another, adapters or linkers can be attached to the fragments.

The production of plant cells with reduced activity of a gene product can be achieved, for example, through the expression of at least one corresponding antisense-RNA, of a sense-RNA for achieving a cosuppression effect or the expression of at least one correspondingly constructed ribozyme which cleaves specific transcripts of the aforementioned gene product.

For this, firstly DNA molecules can be used which include the entire coding sequence of a gene product including any flanking sequences present, and also DNA molecules which only include parts of the coding sequence, in which case it is necessary for these parts to be long enough to bring about an antisense effect in the cells. It is also possible to use DNA sequences which have a high degree of homology to the coding sequences of a gene product, but are not completely identical.

During the expression of nucleic acid molecules in plants, the synthesized protein can be localized in any desired compartment of the plant cell. However, in order to achieve localization in a specific compartment, the coding region can, for example, be linked to DNA sequences which ensure localization in a specific compartment. Sequences of this type are known to the person skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give whole plants. The transgenic plants may in principle be plants of any desired plant species, i.e. both monocotyledonous and also dicotyledonous plants. Thus, transgenic plants are obtainable which have modified properties through overexpression, suppression or inhibition of homologous (=natural) genes or gene sequences or expression of heterologous (=foreign) genes or gene sequences.

When using the compounds of formula (I) in transgenic crops, besides the effects against harmful plants that are observed in other crops, effects often arise which are specific to the application in the particular transgenic crop, for example a modified or specifically expanded broad-leaved weed spectrum which can be controlled, modified application amounts which can be used for the application, preferably good combineability with the herbicides against which the transgenic crop is resistant, and also influencing of growth and yield of the transgenic crop plants. The invention therefore also provides the use of the compounds of formula (I) as herbicides for controlling harmful plants in transgenic crop plants.

Moreover, the compounds of formula (I) have excellent growth-regulatory properties in crop plants. They intervene to regulate the plant's own metabolism and can therefore be used for the targeted influencing of plant ingredients and for ease of harvesting such as, for example, by triggering desiccation and stunted growth. Furthermore, they are also suitable for generally controlling and inhibiting undesired vegetative growth without killing off the plants at the same time. An inhibition of the vegetative growth plays a great role for many mono- and dicotyledonous crops since this allows lodging to be reduced or completely prevented.

The compounds of formula (I) can be formulated in different ways to give herbicidal compositions according to which biological and/or chemical-physical parameters are prescribed. Suitable formulation possibilities are, for example: spray powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, dusting agents (DP), capsule suspensions (CS), seed dressings, granules for scattering and soil application, granules (GR) in the form of microgranules, spray granules, coated granules and adsorption granules, water-dispersible granules (WG), water-soluble granules (SG), ULV formulations, microcapsules and waxes. These individual formulation types are known in principle and are described, for example, in: Winnacker-Küchler, “Chemische Technologie [Chemical Technology]”, volume 7, C. Hanser Verlag Munich, 4th edition, 1986, Wade van Valkenburg, “Pesticide Formulations”, Marcel Dekker, N.Y., 1973; K. Martens, “Spray Drying” Handbook, 3rd Ed., 1979, G. Goodwin Ltd. London. Such herbicidal compositions are likewise provided by the invention.

The necessary formulation auxiliaries such as inert materials, surfactants, solvents and further additives are likewise known and are described, for example, in: Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte [Surface-active ethylene oxide adducts]”, Wiss. Verlagsgesell., Stuttgart 1976; Winnacker-Küchler, “Chemische Technologie [Chemical Technology]”, volume 7, C. Hanser Verlag Munich, 4th edition, 1986.

Spray powders are preparations which can be dispersed uniformly in water and which, besides the active ingredient, apart from a diluent or inert substance, also comprise surfactants of ionic and/or nonionic type (wetting agent, dispersant), e.g. polyoxyethylated alkylphenols, polyoxethylated fatty alcohols, polyoxethylated fatty amines, fatty alcohol polyglycol ether sulfates, alkanesulfonates, alkylbenzene-sulfonates, sodium 2,2′-dinaphthylmethane-6,6′-disulfonate, sodium lignosulfonate, sodium dibutylnaphthalenesulfonate and also sodium oleoylmethyltaurate. To prepare the spray powders, the herbicidal active ingredients are finely ground, for example in customary apparatuses such as hammer mills, blower mills and air-jet mills and are mixed simultaneously or subsequently with the formulation auxiliaries.

Emulsifiable concentrates are prepared by dissolving the active ingredient in an organic solvent, e.g. butanol, cyclohexanone, DMF, xylene or else higher-boiling aromatics or hydrocarbons or mixtures of these solvents with the addition of one or more surfactants of ionic and/or nonionic type (emulsifiers). Emulsifiers which can be used are, for example: alkylarylsulfonic calcium salts, such as Ca dodecylbenzenesulfonate or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters such as, for example, sorbitan fatty acid esters and polyoxethylene sorbitan esters such as, for example, polyoxyethylene sorbitan fatty acid esters.

Dusting agents are obtained by grinding the active ingredient with finely divided solid substances, e.g. talc, natural clays, such as kaolin, bentonite and pyrophyllite, or diatomaceous earth. Suspension concentrates may be water-based or oil-based. They can be prepared, for example, by wet grinding by means of standard commercial bead mills and if appropriate addition of surfactants, as are listed, for example, above in connection with the other types of formulation.

Emulsions, e.g. oil-in-water emulsions (EW), can be prepared, for example, by means of stirrers, colloid mills and/or static mixers using aqueous organic solvents and if appropriate surfactants, as have already been listed above, for example, in connection with the other types of formulation.

Granules can be prepared either by atomizing the active ingredient onto granulated inert material that is capable of adsorption or by applying active ingredient concentrates by means of adhesives, e.g. polyvinyl alcohol, polyacrylic sodium or else mineral oils, onto the surface of carrier substances such as sand, kaolinites or of granulated inert material. Suitable active ingredients can also be granulated in the manner customary for producing fertilizer granules—if desired in a mixture with fertilizers. Water-dispersible granules are usually prepared by customary methods such as spray-drying, fluidized-bed granulation, pan granulation, mixing using high-speed mixers and extrusion without solid inert material.

For the preparation of pan, fluidized-bed, extruder and spray granules, see, for example, methods in “Spray-Drying Handbook” 3rd ed., 1979, G. Goodwin Ltd., London; J. E. Browning, “Agglomeration”, Chemical and Engineering 1967, pages 147 if; “Perry's Chemical Engineer's Handbook”, 5th Ed., McGraw-Hill, New York 1973, pp. 8-57. For further details relating to the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations generally comprise 0.1 to 99% by weight, in particular 0.1 to 95% by weight, of active ingredient of formula (I). In spray powders, the active ingredient concentration is, for example, about 10 to 90% by weight, the remainder to 100% by weight consists of customary formulation constituents. In the case of emulsifiable concentrates, the active ingredient concentration can be about 1 to 90% by weight, preferably 5 to 80% by weight. Dust-like formulations comprise 1 to 30% by weight of active ingredient, preferably at most 5 to 20% by weight of active ingredient, sprayable solutions comprise about 0.05 to 80% by weight, preferably 2 to 50% by weight, of active ingredient. In the case of water-dispersible granules, the active ingredient content depends partly on whether the active compound is present in liquid or solid form and which granulation auxiliaries, fillers, etc. are used. In the case of the water-dispersible granules, the content of active ingredient is, for example, between 1 and 95% by weight, preferably between 10 and 80% by weight.

In addition, the specified active ingredient formulations optionally comprise the adhesives, wetting agents, dispersants, emulsifiers, penetration agents, preservatives, antifreezes and solvents, fillers, carriers and dyes, antifoams, evaporation inhibitors and agents which influence the pH and the viscosity that are customary in each case.

On the basis of these formulations, it is also possible to prepare combinations with other pesticide substances, such as, for example, insecticides, acaricides, herbicides, fungicides, and also with safeners, fertilizers and/or growth regulators, e.g. in the form of a finished formulation or as tank mix.

Combination partners which can be used for the compounds of formula (I) in mixture formulations or in the tank mix are, for example, known active ingredients, as are described, for example, in Weed Research 26, 441-445 (1986) or “The Pesticide Manual”, 13th edition, The British Crop Protection Council and the Royal Soc. of Chemistry, 2003 and literature cited therein. Known herbicides which can be combined with the compounds of formula (I) are, for example, the following active ingredients (note: the compounds are designated either with the “common name” in accordance with the International Organization for Standardization (ISO) or with the chemical name, optionally together with a customary code number):

acetochlor; acifluorfen; aclonifen; AKH 7088, i.e. [[[1-[5-[2-chloro-4-(trifluoromethyl)-phenoxy]-2-nitrophenyl]-2-methoxyethylidene]amino]oxy]acetic acid and its methyl ester; alachlor; alloxydim; ametryn; amicarbazone; amidosulfuron; amitrol; AMS, i.e. ammonium sulfamate; anilofos; asulam; atrazin; azimsulfuron (DPX-A8947); aziprotryn; barban; BAS 516H, i.e. 5-fluoro-2-phenyl-4H-3,1-benzoxazin-4-one; benazolin; benfluralin; benfuresate; bensulfuron-methyl; bensulide; bentazone; benzfenap; benzofluor; benzoylprop-ethyl; benzthiazuron; bialaphos; bifenox; bromacil; bromobutide; bromofenoxim; bromoxynil; bromuron; buminafos; busoxinone; butachlor; butamifos; butenachlor; buthidazole; butralin; butylate; cafenstrole (CH-900); carbetamide; cafentrazone (ICI-A0051); CDAA, i.e. 2-chloro-N,N-di-2-propenylacetamide; CDEC, i.e. 2-chloroallyl diethyldithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron ethyl; chlornitrofen; chlorotoluron; chloroxuron; chlorpropham; chlorsulfuron; chlorthal-dimethyl; chlorthiamid; cinmethylin; cinosulfuron; clethodim; clodinafop and ester derivatives thereof (e.g. clodinafop-propargyl); clomazone; clomeprop; cloproxydim; clopyralid; cumyluron (JC 940); cyanazine; cycloate; cyclosulfamuron (AC 104); cycloxydim; cycluron; cyhalofop and ester derivatives thereof (e.g. butyl ester, DEH-112); cyperquat; cyprazine; cyprazole; daimuron; 2,4-DB; dalapon; desmedipham; desmetryn; di-allate; dicamba; dichlobenil; dichlorprop; diclofop and esters thereof such as diclofop-methyl; diethatyl; difenoxuron; difenzoquat; diflufenican; dimefuron; dimethachlor; dimethametryn; dimethenamid (SAN-582H); dimethazone, clomazon; dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; EPTC; esprocarb; ethalfluralin; ethametsulfuron-methyl; ethidimuron; ethiozin; ethofumesate; F5231, i.e. N-[2-chloro-4-fluoro-5-[4-(3-fluoropropyl)-4,5-dihydro-5-oxo-1H-tetrazol-1-yl]phenyl]ethanesulfonamide; ethoxyfen and esters thereof (e.g. ethyl ester, HN-252); etobenzanid (HW 52); fenoprop; fenoxan, fenoxapropand fenoxaprop-P and esters thereof, e.g. fenoxaprop-P-ethyl and fenoxaprop-ethyl; fenoxydim; fenuron; flamprop-methyl; flazasulfuron; fluazifop and fluazifop-P and esters thereof, e.g. fluazifop-butyl and fluazifop-P-butyl; fluchloralin; flumetsulam; flumeturon; flumiclorac and esters thereof (e.g. pentyl ester, S-23031); flumioxazin (S-482); flumipropyn; flupoxam (KNW-739); fluorodifen; fluoroglycofen-ethyl; flupropacil (UBIC-4243); fluridone; fluorochloridone; fluoroxypyr; flurtamone; fomesafen; fosamine; furyloxyfen; glufosinate; glyphosate; halosafen; halosulfuron and esters thereof (e.g. methyl ester, NC-319); haloxyfop and esters thereof; haloxyfop-P (=R-haloxyfop) and esters thereof; hexazinone; imazapyr; imazamethabenz-methyl; imazaquin and salts such as the ammonium salt; ioxynil; imazethamethapyr; imazethapyr; imazosulfuron; isocarbamid; isopropalin; isoproturon; isouron; isoxaben; isoxapyrifop; karbutilate; lactofen; lenacil; linuron; MCPA; MCPB; mecoprop; mefenacet; mefluidid; metamitron; metazachlor; metham; methabenzthiazuron; methazole; methoxyphenone; methyldymron; metabenzuron, methobenzuron; metobromuron; metolachlor; metosulam (XRD 511); metoxuron; metribuzin; metsulfuron-methyl; MH; molinate; monalide; monolinuron; monuron; monocarbamide dihydrogensulfate; MT 128, i.e. 6-chloro-N-(3-chloro-2-propenyl)-5-methyl-N-phenyl-3-pyridazinamine; MT 5950, i.e. N-[3-chloro-4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazon; oxyfluorfen; paraquat; pebulate; pendimethalin; perfluidone; phenisopham; phenmedipham; picloram; pinoxaden; piperophos; piributicarb; pirifenop-butyl; pretilachlor; primisulfuron-methyl; procyazine; prodiamine; profluralin; proglinazine-ethyl; prometon; prometryn; propachlor; propanil; propaquizafop and esters thereof; propazine; propham; propisochlor; propoxycarbazone; propyzamide; prosulfalin; prosulfocarb; prosulfuron (CGA-152005); prynachlor; pyraclonil, pyrazolinate; pyrazon; pyrazosulfuron-ethyl; pyrazoxyfen; pyridate; pyrithiobac (KIH-2031); pyroxofop and esters thereof (e.g. propargyl ester); quinclorac; quinmerac; quinofop and ester derivatives thereof, quizalofop and quizalofop-P ester derivatives thereof, e.g. quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and methyl ester; sulfentrazon (FMC-97285, F-6285); sulfazuron; sulfometuron-methyl; sulfosate (ICI-A0224); TCA; tebutam (GCP-5544); tebuthiuron; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiazafluoron; thiazopyr (Mon-13200); thidiazimin (SN-24085); thiobencarb; thifensulfuron-methyl; tiocarbazil; tralkoxydim; tri-allate; triasulfuron; triazofenamide; tribenuron-methyl; triclopyr; tridiphane; trietazine; trifluralin; triflusulfuron and esters (e.g. methyl ester, DPX-66037); trimeturon; tsitodef; vernolate; WL 110547, i.e. 5-phenoxy-1-[3-(trifluoromethyl)phenyl]-1H-tetrazole; UBH-509; D-489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127; KIH-2023 and KIH-485.

For use, the formulations present in standard commercial form are optionally diluted in the usual manner, e.g. in the case of spray powders, emulsifiable concentrates, dispersions and water-dispersible granules by means of water. Dust-like preparations, soil and scatter granules and also sprayable solutions are usually not diluted with further inert substances prior to use. The required rate of application of the compounds of formula (I) varies inter alia with the external conditions such as temperature, humidity, type of herbicide used. It can vary within wide limits, e.g. between 0.001 and 1.0 kg/ha or more active substance, but is preferably between 5 and 750 g/ha, in particular between 5 and 250 g/ha.

The examples below illustrate the invention.

The abbreviations used here mean:

| | | |
|---|---|---|
| iPr = isopropyl | cPr = cyclopropyl | Pr = propyl |
| Et = ethyl | Me = methyl | Ph = phenyl |
| tBu = tertiary-butyl | | |

TABLE 1
Compounds of formula (Ia) [═ compounds according to the invention of
general formula (I) in which R⁴ is methyl]

| | |
|---|---|
| | (Ia) |
| 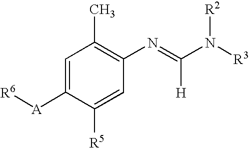 | |

| No. | R² | R³ | R⁵ | A | R⁶ |
|---|---|---|---|---|---|
| 1 | Me | Me | Me | O | 3-Br-5-F-Phenyl |
| 2 | Me | Et | Me | O | 3-Br-5-F-Phenyl |
| 3 | (CH₂)₄ | | Me | O | 3-Br-5-F-Phenyl |
| 4 | (CH₂)₅ | | Me | O | 3-Br-5-F-Phenyl |
| 5 | Me | Me | Me | S | 3-Br-5-F-Phenyl |
| 6 | Me | Et | Me | S | 3-Br-5-F-Phenyl |
| 7 | (CH₂)₄ | | Me | S | 3-Br-5-F-Phenyl |
| 8 | (CH₂)₅ | | Me | S | 3-Br-5-F-Phenyl |
| 9 | Me | Me | Me | CH₂CH₂ | 3-Br-5-F-Phenyl |
| 10 | Me | Et | Me | CH₂CH₂ | 3-Br-5-F-Phenyl |
| 11 | (CH₂)₄ | | Me | CH₂CH₂ | 3-Br-5-F-Phenyl |
| 12 | (CH₂)₅ | | Me | CH₂CH₂ | 3-Br-5-F-Phenyl |
| 13 | Me | Me | Me | CH₂═CH₂ | 3-Br-5-F-Phenyl |
| 14 | Me | Et | Me | CH₂═CH₂ | 3-Br-5-F-Phenyl |
| 15 | (CH₂)₄ | | Me | CH₂═CH₂ | 3-Br-5-F-Phenyl |
| 16 | (CH₂)₅ | | Me | CH₂═CH₂ | 3-Br-5-F-Phenyl |
| 17 | Me | Me | Me | O | 4-Br-3-iPr-Phenyl |
| 18 | Me | Et | Me | O | 4-Br-3-iPr-Phenyl |
| 19 | (CH₂)₄ | | Me | O | 4-Br-3-iPr-Phenyl |
| 20 | (CH₂)₅ | | Me | O | 4-Br-3-iPr-Phenyl |
| 21 | Me | Me | Me | S | 4-Br-3-iPr-Phenyl |
| 22 | Me | Et | Me | S | 4-Br-3-iPr-Phenyl |
| 23 | (CH₂)₄ | | Me | S | 4-Br-3-iPr-Phenyl |
| 24 | (CH₂)₅ | | Me | S | 4-Br-3-iPr-Phenyl |
| 25 | Me | Me | Me | CH₂CH₂ | 4-Br-3-iPr-Phenyl |
| 26 | Me | Et | Me | CH₂CH₂ | 4-Br-3-iPr-Phenyl |
| 27 | (CH₂)₄ | | Me | CH₂CH₂ | 4-Br-3-iPr-Phenyl |
| 28 | (CH₂)₅ | | Me | CH₂CH₂ | 4-Br-3-iPr-Phenyl |
| 29 | Me | Me | Me | CH₂═CH₂ | 4-Br-3-iPr-Phenyl |
| 30 | Me | Et | Me | CH₂═CH₂ | 4-Br-3-iPr-Phenyl |
| 31 | (CH₂)₄ | | Me | CH₂═CH₂ | 4-Br-3-iPr-Phenyl |
| 32 | (CH₂)₅ | | Me | CH₂═CH₂ | 4-Br-3-iPr-Phenyl |
| 33 | Me | Me | Me | O | 4-Cl-3-CF₃-Phenyl |
| 34 | Me | Et | Me | O | 4-Cl-3-CF₃-Phenyl |
| 35 | (CH₂)₄ | | Me | O | 4-Cl-3-CF₃-Phenyl |
| 36 | (CH₂)₅ | | Me | O | 4-Cl-3-CF₃-Phenyl |
| 37 | Me | Me | Me | S | 4-Cl-3-CF₃-Phenyl |
| 38 | Me | Et | Me | S | 4-Cl-3-CF₃-Phenyl |
| 39 | (CH₂)₄ | | Me | S | 4-Cl-3-CF₃-Phenyl |
| 40 | (CH₂)₅ | | Me | S | 4-Cl-3-CF₃-Phenyl |
| 41 | Me | Me | Me | CH₂CH₂ | 4-Cl-3-CF₃-Phenyl |
| 42 | Me | Et | Me | CH₂CH₂ | 4-Cl-3-CF₃-Phenyl |
| 43 | (CH₂)₄ | | Me | CH₂CH₂ | 4-Cl-3-CF₃-Phenyl |
| 44 | (CH₂)₅ | | Me | CH₂CH₂ | 4-Cl-3-CF₃-Phenyl |
| 45 | Me | Me | Me | CH₂═CH₂ | 4-Cl-3-CF₃-Phenyl |
| 46 | Me | Et | Me | CH₂═CH₂ | 4-Cl-3-CF₃-Phenyl |
| 47 | (CH₂)₄ | | Me | CH₂═CH₂ | 4-Cl-3-CF₃-Phenyl |
| 48 | (CH₂)₅ | | Me | CH₂═CH₂ | 4-Cl-3-CF₃-Phenyl |
| 49 | Me | Me | Me | O | 4-Cl-3-tBu-Phenyl |
| 50 | Me | Et | Me | O | 4-Cl-3-tBu-Phenyl |
| 51 | (CH₂)₄ | | Me | O | 4-Cl-3-tBu-Phenyl |
| 52 | (CH₂)₄ | | Me | O | 4-Cl-3-tBu-Phenyl |
| 53 | Me | Me | Me | S | 4-Cl-3-tBu-Phenyl |
| 54 | Me | Et | Me | S | 4-Cl-3-tBu-Phenyl |
| 55 | (CH₂)₄ | | Me | S | 4-Cl-3-tBu-Phenyl |
| 56 | (CH₂)₅ | | Me | S | 4-Cl-3-tBu-Phenyl |
| 57 | Me | Me | Me | CH₂CH₂ | 4-Cl-3-tBu-Phenyl |
| 58 | Me | Et | Me | CH₂CH₂ | 4-Cl-3-tBu-Phenyl |
| 59 | (CH₂)₄ | | Me | CH₂CH₂ | 4-Cl-3-tBu-Phenyl |
| 60 | (CH₂)₅ | | Me | CH₂CH₂ | 4-Cl-3-tBu-Phenyl |
| 61 | Me | Me | Me | CH₂═CH₂ | 4-Cl-3-tBu-Phenyl |
| 62 | Me | Et | Me | CH₂═CH₂ | 4-Cl-3-tBu-Phenyl |
| 63 | (CH₂)₄ | | Me | CH₂═CH₂ | 4-Cl-3-tBu-Phenyl |
| 64 | (CH₂)₅ | | Me | CH₂═CH₂ | 4-Cl-3-tBu-Phenyl |
| 65 | Me | Me | Me | O | 4-Cl-2-Me-Phenyl |
| 66 | Me | Et | Me | O | 4-Cl-2-Me-Phenyl |
| 67 | (CH₂)₄ | | Me | O | 4-Cl-2-Me-Phenyl |
| 68 | (CH₂)₅ | | Me | O | 4-Cl-2-Me-Phenyl |
| 69 | Me | Me | Me | S | 4-Cl-2-Me-Phenyl |
| 70 | Me | Et | Me | S | 4-Cl-2-Me-Phenyl |
| 71 | (CH₂)₄ | | Me | S | 4-Cl-2-Me-Phenyl |
| 72 | (CH₂)₅ | | Me | S | 4-Cl-2-Me-Phenyl |
| 73 | Me | Me | Me | CH₂CH₂ | 4-Cl-2-Me-Phenyl |
| 74 | Me | Et | Me | CH₂CH₂ | 4-Cl-2-Me-Phenyl |
| 75 | (CH₂)₄ | | Me | CH₂CH₂ | 4-Cl-2-Me-Phenyl |
| 76 | (CH₂)₅ | | Me | CH₂CH₂ | 4-Cl-2-Me-Phenyl |
| 77 | Me | Me | Me | CH₂═CH₂ | 4-Cl-2-Me-Phenyl |
| 78 | Me | Et | Me | CH₂═CH₂ | 4-Cl-2-Me-Phenyl |
| 79 | (CH₂)₄ | | Me | CH₂═CH₂ | 4-Cl-2-Me-Phenyl |
| 80 | (CH₂)₅ | | Me | CH₂═CH₂ | 4-Cl-2-Me-Phenyl |
| 81 | Me | Me | Me | O | 4-Cl-3-Me-Phenyl |
| 82 | Me | Et | Me | O | 4-Cl-3-Me-Phenyl |
| 83 | (CH₂)₄ | | Me | O | 4-Cl-3-Me-Phenyl |
| 84 | (CH₂)₅ | | Me | O | 4-Cl-3-Me-Phenyl |
| 85 | Me | Me | Me | S | 4-Cl-3-Me-Phenyl |
| 86 | Me | Et | Me | S | 4-Cl-3-Me-Phenyl |
| 87 | (CH₂)₄ | | Me | S | 4-Cl-3-Me-Phenyl |
| 88 | (CH₂)₅ | | Me | S | 4-Cl-3-Me-Phenyl |
| 89 | Me | Me | Me | CH₂CH₂ | 4-Cl-3-Me-Phenyl |
| 90 | Me | Et | Me | CH₂CH₂ | 4-Cl-3-Me-Phenyl |
| 91 | (CH₂)₄ | | Me | CH₂CH₂ | 4-Cl-3-Me-Phenyl |
| 92 | (CH₂)₅ | | Me | CH₂CH₂ | 4-Cl-3-Me-Phenyl |
| 93 | Me | Me | Me | CH₂═CH₂ | 4-Cl-3-Me-Phenyl |
| 94 | Me | Et | Me | CH₂═CH₂ | 4-Cl-3-Me-Phenyl |
| 95 | (CH₂)₄ | | Me | CH₂═CH₂ | 4-Cl-3-Me-Phenyl |
| 96 | (CH₂)₅ | | Me | CH₂═CH₂ | 4-Cl-3-Me-Phenyl |
| 97 | Me | Me | Me | O | 4-Cl-3-iPr-Phenyl |
| 98 | Me | Et | Me | O | 4-Cl-3-iPr-Phenyl |
| 99 | (CH₂)₄ | | Me | O | 4-Cl-3-iPr-Phenyl |
| 100 | (CH₂)₅ | | Me | O | 4-Cl-3-iPr-Phenyl |
| 101 | Me | Me | Me | S | 4-Cl-3-iPr-Phenyl |
| 102 | Me | Et | Me | S | 4-Cl-3-iPr-Phenyl |
| 103 | (CH₂)₄ | | Me | S | 4-Cl-3-iPr-Phenyl |
| 104 | (CH₂)₅ | | Me | S | 4-Cl-3-iPr-Phenyl |
| 105 | Me | Me | Me | CH₂CH₂ | 4-Cl-3-iPr-Phenyl |
| 106 | Me | Et | Me | CH₂CH₂ | 4-Cl-3-iPr-Phenyl |
| 107 | (CH₂)₄ | | Me | CH₂CH₂ | 4-Cl-3-iPr-Phenyl |
| 108 | (CH₂)₅ | | Me | CH₂CH₂ | 4-Cl-3-iPr-Phenyl |
| 109 | Me | Me | Me | CH₂═CH₂ | 4-Cl-3-iPr-Phenyl |
| 110 | Me | Et | Me | CH₂═CH₂ | 4-Cl-3-iPr-Phenyl |
| 111 | (CH₂)₄ | | Me | CH₂═CH₂ | 4-Cl-3-iPr-Phenyl |
| 112 | (CH₂)₅ | | Me | CH₂═CH₂ | 4-Cl-3-iPr-Phenyl |
| 113 | Me | Me | Me | O | 4-Cl-6-iPr-Pyridin-2-yl |
| 114 | Me | Et | Me | O | 4-Cl-6-iPr-Pyridin-2-yl |
| 115 | (CH₂)₄ | | Me | O | 4-Cl-6-iPr-Pyridin-2-yl |
| 116 | (CH₂)₅ | | Me | O | 4-Cl-6-iPr-Pyridin-2-yl |
| 117 | Me | Me | Me | S | 4-Cl-6-iPr-Pyridin-2-yl |
| 118 | Me | Et | Me | S | 4-Cl-6-iPr-Pyridin-2-yl |
| 119 | (CH₂)₄ | | Me | S | 4-Cl-6-iPr-Pyridin-2-yl |
| 120 | (CH₂)₅ | | Me | S | 4-Cl-6-iPr-Pyridin-2-yl |
| 121 | Me | Me | Me | OCH₂ | 4-Cl-6-iPr-Pyridin-2-yl |
| 122 | Me | Et | Me | OCH₂ | 4-Cl-6-iPr-Pyridin-2-yl |
| 123 | (CH₂)₄ | | Me | OCH₂ | 4-Cl-6-iPr-Pyridin-2-yl |
| 124 | (CH₂)₅ | | Me | OCH₂ | 4-Cl-6-iPr-Pyridin-2-yl |
| 125 | Me | Me | Me | CH₂═CH₂ | 4-Cl-6-iPr-Pyridin-2-yl |
| 126 | Me | Et | Me | CH₂═CH₂ | 4-Cl-6-iPr-Pyridin-2-yl |
| 127 | (CH₂)₄ | | Me | CH₂═CH₂ | 4-Cl-6-iPr-Pyridin-2-yl |
| 128 | (CH₂)₅ | | Me | CH₂═CH₂ | 4-Cl-6-iPr-Pyridin-2-yl |
| 129 | Me | Me | Me | O | 3-(2-Cl-Pyridin-3-yl)-Phenyl |
| 130 | Me | Et | Me | O | 3-(2-Cl-Pyridin-3-yl)-Phenyl |
| 131 | (CH₂)₄ | | Me | O | 3-(2-Cl-Pyridin-3-yl)-Phenyl |
| 132 | (CH₂)₅ | | Me | O | 3-(2-Cl-Pyridin-3-yl)-Phenyl |
| 133 | Me | Me | Me | S | 3-(2-Cl-Pyridin-3-yl)-Phenyl |
| 134 | Me | Et | Me | S | 3-(2-Cl-Pyridin-3-yl)-Phenyl |
| 135 | (CH₂)₄ | | Me | S | 3-(2-Cl-Pyridin-3-yl)-Phenyl |
| 136 | (CH₂)₅ | | Me | S | 3-(2-Cl-Pyridin-3-yl)-Phenyl |
| 137 | Me | Me | Me | OCH₂ | 2-Cl-Pyridin-3-yl |
| 138 | Me | Et | Me | OCH₂ | 2-Cl-Pyridin-3-yl |
| 139 | (CH₂)₄ | | Me | OCH₂ | 2-Cl-Pyridin-3-yl |
| 140 | (CH₂)₅ | | Me | OCH₂ | 2-Cl-Pyridin-3-yl |
| 141 | Me | Me | Me | CH₂═CH₂ | 2-Cl-Pyridin-3-yl |
| 142 | Me | Et | Me | CH₂═CH₂ | 2-Cl-Pyridin-3-yl |
| 143 | (CH₂)₄ | | Me | CH₂═CH₂ | 2-Cl-Pyridin-3-yl |
| 144 | (CH₂)₅ | | Me | CH₂═CH₂ | 2-Cl-Pyridin-3-yl |
| 145 | Me | Me | Me | O | 3-CF₃-4-Me-Phenyl |
| 146 | Me | Et | Me | O | 3-CF₃-4-Me-Phenyl |
| 147 | (CH₂)₄ | | Me | O | 3-CF₃-4-Me-Phenyl |
| 148 | (CH₂)₅ | | Me | O | 3-CF₃-4-Me-Phenyl |
| 149 | Me | Me | Me | S | 3-CF₃-4-Me-Phenyl |
| 150 | Me | Et | Me | S | 3-CF₃-4-Me-Phenyl |
| 151 | (CH₂)₄ | | Me | S | 3-CF₃-4-Me-Phenyl |
| 152 | (CH₂)₅ | | Me | S | 3-CF₃-4-Me-Phenyl |
| 153 | Me | Me | Me | CH₂CH₂ | 3-CF₃-4-Me-Phenyl |
| 154 | Me | Et | Me | CH₂CH₂ | 3-CF₃-4-Me-Phenyl |
| 155 | (CH₂)₄ | | Me | CH₂CH₂ | 3-CF₃-4-Me-Phenyl |
| 156 | (CH₂)₅ | | Me | CH₂CH₂ | 3-CF₃-4-Me-Phenyl |
| 157 | Me | Me | Me | CH₂═CH₂ | 3-CF₃-4-Me-Phenyl |
| 158 | Me | Et | Me | CH₂═CH₂ | 3-CF₃-4-Me-Phenyl |
| 159 | (CH₂)₄ | | Me | CH₂═CH₂ | 3-CF₃-4-Me-Phenyl |
| 160 | (CH₂)₅ | | Me | CH₂═CH₂ | 3-CF₃-4-Me-Phenyl |
| 161 | Me | Me | Me | O | 4-F-3-OCF₃-Phenyl |
| 162 | Me | Et | Me | O | 4-F-3-OCF₃-Phenyl |
| 163 | (CH₂)₄ | | Me | O | 4-F-3-OCF₃-Phenyl |
| 164 | (CH₂)₅ | | Me | O | 4-F-3-OCF₃-Phenyl |
| 165 | Me | Me | Me | S | 4-F-3-OCF₃-Phenyl |
| 166 | Me | Et | Me | S | 4-F-3-OCF₃-Phenyl |
| 167 | (CH₂)₄ | | Me | S | 4-F-3-OCF₃-Phenyl |
| 168 | (CH₂)₅ | | Me | S | 4-F-3-OCF₃-Phenyl |
| 169 | Me | Me | Me | CH₂CH₂ | 4-F-3-OCF₃-Phenyl |
| 170 | Me | Et | Me | CH₂CH₂ | 4-F-3-OCF₃-Phenyl |
| 171 | (CH₂)₄ | | Me | CH₂CH₂ | 4-F-3-OCF₃-Phenyl |
| 172 | (CH₂)₅ | | Me | CH₂CH₂ | 4-F-3-OCF₃-Phenyl |
| 173 | Me | Me | Me | CH₂═CH₂ | 4-F-3-OCF₃-Phenyl |
| 174 | Me | Et | Me | CH₂═CH₂ | 4-F-3-OCF₃-Phenyl |
| 175 | (CH₂)₄ | | Me | CH₂═CH₂ | 4-F-3-OCF₃-Phenyl |
| 176 | (CH₂)₅ | | Me | CH₂═CH₂ | 4-F-3-OCF₃-Phenyl |
| 177 | Me | Me | Me | O | 3-I-5-CF₃-Phenyl |
| 178 | Me | Et | Me | O | 3-I-5-CF₃-Phenyl |
| 179 | (CH₂)₄ | | Me | O | 3-I-5-CF₃-Phenyl |
| 180 | (CH₂)₅ | | Me | O | 3-I-5-CF₃-Phenyl |
| 181 | Me | Me | Me | S | 3-I-5-CF₃-Phenyl |
| 182 | Me | Et | Me | S | 3-I-5-CF₃-Phenyl |
| 183 | (CH₂)₄ | | Me | S | 3-I-5-CF₃-Phenyl |
| 184 | (CH₂)₅ | | Me | S | 3-I-5-CF₃-Phenyl |
| 185 | Me | Me | Me | CH₂CH₂ | 3-I-5-CF₃-Phenyl |
| 186 | Me | Et | Me | CH₂CH₂ | 3-I-5-CF₃-Phenyl |
| 187 | (CH₂)₄ | | Me | CH₂CH₂ | 3-I-5-CF₃-Phenyl |
| 188 | (CH₂)₅ | | Me | CH₂CH₂ | 3-I-5-CF₃-Phenyl |
| 189 | Me | Me | Me | CH₂═CH₂ | 3-I-5-CF₃-Phenyl |
| 190 | Me | Et | Me | CH₂═CH₂ | 3-I-5-CF₃-Phenyl |
| 191 | (CH₂)₄ | | Me | CH₂═CH₂ | 3-I-5-CF₃-Phenyl |
| 192 | (CH₂)₅ | | Me | CH₂═CH₂ | 3-I-5-CF₃-Phenyl |
| 193 | Me | Me | Me | O | 3-tBu-Phenyl |
| 194 | Me | Et | Me | O | 3-tBu-Phenyl |
| 195 | (CH₂)₄ | | Me | O | 3-tBu-Phenyl |
| 196 | (CH₂)₅ | | Me | O | 3-tBu-Phenyl |
| 197 | Me | Me | Me | S | 3-tBu-Phenyl |
| 198 | Me | Et | Me | S | 3-tBu-Phenyl |
| 199 | (CH₂)₄ | | Me | S | 3-tBu-Phenyl |
| 200 | (CH₂)₅ | | Me | S | 3-tBu-Phenyl |
| 201 | Me | Me | Me | CH₂CH₂ | 3-tBu-Phenyl |
| 202 | Me | Et | Me | CH₂CH₂ | 3-tBu-Phenyl |
| 203 | (CH₂)₄ | | Me | CH₂CH₂ | 3-tBu-Phenyl |
| 204 | (CH₂)₅ | | Me | CH₂CH₂ | 3-tBu-Phenyl |
| 205 | Me | Me | Me | CH₂═CH₂ | 3-tBu-Phenyl |
| 206 | Me | Et | Me | CH₂═CH₂ | 3-tBu-Phenyl |
| 207 | (CH₂)₄ | | Me | CH₂═CH₂ | 3-tBu-Phenyl |
| 208 | (CH₂)₅ | | Me | CH₂═CH₂ | 3-tBu-Phenyl |
| 209 | Me | Me | Me | O | 4-tBu-Phenyl |
| 210 | Me | Et | Me | O | 4-tBu-Phenyl |
| 211 | (CH₂)₄ | | Me | O | 4-tBu-Phenyl |
| 212 | (CH₂)₅ | | Me | O | 4-tBu-Phenyl |
| 213 | Me | Me | Me | S | 4-tBu-Phenyl |
| 214 | Me | Et | Me | S | 4-tBu-Phenyl |
| 215 | (CH₂)₄ | | Me | S | 4-tBu-Phenyl |
| 216 | (CH₂)₅ | | Me | S | 4-tBu-Phenyl |
| 217 | Me | Me | Me | CH₂CH₂ | 4-tBu-Phenyl |
| 218 | Me | Et | Me | CH₂CH₂ | 4-tBu-Phenyl |
| 219 | (CH₂)₄ | | Me | CH₂CH₂ | 4-tBu-Phenyl |
| 220 | (CH₂)₅ | | Me | CH₂CH₂ | 4-tBu-Phenyl |
| 221 | Me | Me | Me | CH₂═CH₂ | 4-tBu-Phenyl |
| 222 | Me | Et | Me | CH₂═CH₂ | 4-tBu-Phenyl |
| 223 | (CH₂)₄ | | Me | CH₂═CH₂ | 4-tBu-Phenyl |
| 224 | (CH₂)₅ | | Me | CH₂═CH₂ | 4-tBu-Phenyl |
| 225 | Me | Me | Me | O | 4-CN-3-CF₃-Phenyl |
| 226 | Me | Et | Me | O | 4-CN-3-CF₃-Phenyl |
| 227 | (CH₂)₄ | | Me | O | 4-CN-3-CF₃-Phenyl |
| 228 | (CH₂)₅ | | Me | O | 4-CN-3-CF₃-Phenyl |
| 229 | Me | Me | Me | S | 4-CN-3-CF₃-Phenyl |
| 230 | Me | Et | Me | S | 4-CN-3-CF₃-Phenyl |
| 231 | (CH₂)₄ | | Me | S | 4-CN-3-CF₃-Phenyl |
| 232 | (CH₂)₅ | | Me | S | 4-CN-3-CF₃-Phenyl |
| 233 | Me | Me | Me | CH₂CH₂ | 4-CN-3-CF₃-Phenyl |
| 234 | Me | Et | Me | CH₂CH₂ | 4-CN-3-CF₃-Phenyl |
| 235 | (CH₂)₄ | | Me | CH₂CH₂ | 4-CN-3-CF₃-Phenyl |
| 236 | (CH₂)₅ | | Me | CH₂CH₂ | 4-CN-3-CF₃-Phenyl |
| 237 | Me | Me | Me | CH₂═CH₂ | 4-CN-3-CF₃-Phenyl |
| 238 | Me | Et | Me | CH₂═CH₂ | 4-CN-3-CF₃-Phenyl |
| 239 | (CH₂)₄ | | Me | CH₂═CH₂ | 4-CN-3-CF₃-Phenyl |
| 240 | (CH₂)₅ | | Me | CH₂═CH₂ | 4-CN-3-CF₃-Phenyl |
| 241 | Me | Me | Me | O | 4-CF₃-Phenyl |
| 242 | Me | Et | Me | O | 4-CF₃-Phenyl |
| 243 | (CH₂)₄ | | Me | O | 4-CF₃-Phenyl |
| 244 | (CH₂)₅ | | Me | O | 4-CF₃-Phenyl |
| 245 | Me | Me | Me | S | 4-CF₃-Phenyl |
| 246 | Me | Et | Me | S | 4-CF₃-Phenyl |
| 247 | (CH₂)₄ | | Me | S | 4-CF₃-Phenyl |
| 248 | (CH₂)₅ | | Me | S | 4-CF₃-Phenyl |
| 249 | Me | Me | Me | CH₂CH₂ | 4-CF₃-Phenyl |
| 250 | Me | Et | Me | CH₂CH₂ | 4-CF₃-Phenyl |
| 251 | (CH₂)₄ | | Me | CH₂CH₂ | 4-CF₃-Phenyl |
| 252 | (CH₂)₅ | | Me | CH₂CH₂ | 4-CF₃-Phenyl |
| 253 | Me | Me | Me | CH₂═CH₂ | 4-CF₃-Phenyl |
| 254 | Me | Et | Me | CH₂═CH₂ | 4-CF₃-Phenyl |
| 255 | (CH₂)₄ | | Me | CH₂═CH₂ | 4-CF₃-Phenyl |
| 256 | (CH₂)₅ | | Me | CH₂═CH₂ | 4-CF₃-Phenyl |
| 257 | Me | Me | Me | O | 2-Me-Phenyl |
| 258 | Me | Et | Me | O | 2-Me-Phenyl |
| 259 | (CH₂)₄ | | Me | O | 2-Me-Phenyl |
| 260 | (CH₂)₅ | | Me | O | 2-Me-Phenyl |
| 261 | Me | Me | Me | S | 2-Me-Phenyl |
| 262 | Me | Et | Me | S | 2-Me-Phenyl |
| 263 | (CH₂)₄ | | Me | S | 2-Me-Phenyl |
| 264 | (CH₂)₅ | | Me | S | 2-Me-Phenyl |
| 265 | Me | Me | Me | CH₂CH₂ | 2-Me-Phenyl |
| 266 | Me | Et | Me | CH₂CH₂ | 2-Me-Phenyl |
| 267 | (CH₂)₄ | | Me | CH₂CH₂ | 2-Me-Phenyl |
| 268 | (CH₂)₅ | | Me | CH₂CH₂ | 2-Me-Phenyl |
| 269 | Me | Me | Me | CH₂═CH₂ | 2-Me-Phenyl |
| 270 | Me | Et | Me | CH₂═CH₂ | 2-Me-Phenyl |
| 271 | (CH₂)₄ | | Me | CH₂═CH₂ | 2-Me-Phenyl |
| 272 | (CH₂)₅ | | Me | CH₂═CH₂ | 2-Me-Phenyl |
| 273 | Me | Me | Me | O | 4-MeO-Phenyl |
| 274 | Me | Et | Me | O | 4-MeO-Phenyl |
| 275 | (CH₂)₄ | | Me | O | 4-MeO-Phenyl |
| 276 | (CH₂)₅ | | Me | O | 4-MeO-Phenyl |
| 277 | Me | Me | Me | S | 4-MeO-Phenyl |
| 278 | Me | Et | Me | S | 4-MeO-Phenyl |
| 279 | (CH₂)₄ | | Me | S | 4-MeO-Phenyl |
| 280 | (CH₂)₅ | | Me | S | 4-MeO-Phenyl |
| 281 | Me | Me | Me | CH₂CH₂ | 4-MeO-Phenyl |
| 282 | Me | Et | Me | CH₂CH₂ | 4-MeO-Phenyl |
| 283 | (CH₂)₄ | | Me | CH₂CH₂ | 4-MeO-Phenyl |
| 284 | (CH₂)₅ | | Me | CH₂CH₂ | 4-MeO-Phenyl |
| 285 | Me | Me | Me | CH₂═CH₂ | 4-MeO-Phenyl |
| 286 | Me | Et | Me | CH₂═CH₂ | 4-MeO-Phenyl |
| 287 | (CH₂)₄ | | Me | CH₂═CH₂ | 4-MeO-Phenyl |
| 288 | (CH₂)₅ | | Me | CH₂═CH₂ | 4-MeO-Phenyl |
| 289 | Me | Me | Me | O | 3-Ph-O-Phenyl |
| 290 | Me | Et | Me | O | 3-Ph-O-Phenyl |
| 291 | (CH₂)₄ | | Me | O | 3-Ph-O-Phenyl |
| 292 | (CH₂)₅ | | Me | O | 3-Ph-O-Phenyl |
| 293 | Me | Me | Me | S | 3-Ph-O-Phenyl |
| 294 | Me | Et | Me | S | 3-Ph-O-Phenyl |
| 295 | (CH₂)₄ | | Me | S | 3-Ph-O-Phenyl |
| 296 | (CH₂)₅ | | Me | S | 3-Ph-O-Phenyl |
| 297 | Me | Me | Me | CH₂CH₂ | 3-Ph-O-Phenyl |
| 298 | Me | Et | Me | CH₂CH₂ | 3-Ph-O-Phenyl |
| 299 | (CH₂)₄ | | Me | CH₂CH₂ | 3-Ph-O-Phenyl |
| 300 | (CH₂)₅ | | Me | CH₂CH₂ | 3-Ph-O-Phenyl |
| 301 | Me | Me | Me | CH₂═CH₂ | 3-Ph-O-Phenyl |
| 302 | Me | Et | Me | CH₂═CH₂ | 3-Ph-O-Phenyl |
| 303 | (CH₂)₄ | | Me | CH₂═CH₂ | 3-Ph-O-Phenyl |
| 304 | (CH₂)₅ | | Me | CH₂═CH₂ | 3-Ph-O-Phenyl |
| 305 | Me | Me | Me | O | 3-(EtO-C(Me)Pr)-Phenyl |
| 306 | Me | Et | Me | O | 3-(EtO-C(Me)Pr)-Phenyl |
| 307 | (CH₂)₄ | | Me | O | 3-(EtO-C(Me)Pr)-Phenyl |
| 308 | (CH₂)₅ | | Me | O | 3-(EtO-C(Me)Pr)-Phenyl |
| 309 | Me | Me | Me | S | 3-(EtO-C(Me)Pr)-Phenyl |
| 310 | Me | Et | Me | S | 3-(EtO-C(Me)Pr)-Phenyl |
| 311 | (CH₂)₄ | | Me | S | 3-(EtO-C(Me)Pr)-Phenyl |
| 312 | (CH₂)₅ | | Me | S | 3-(EtO-C(Me)Pr)-Phenyl |
| 313 | Me | Me | Me | CH₂CH₂ | 3-(EtO-C(Me)Pr)-Phenyl |
| 314 | Me | Et | Me | CH₂CH₂ | 3-(EtO-C(Me)Pr)-Phenyl |
| 315 | (CH₂)₄ | | Me | CH₂CH₂ | 3-(EtO-C(Me)Pr)-Phenyl |
| 316 | (CH₂)₅ | | Me | CH₂CH₂ | 3-(EtO-C(Me)Pr)-Phenyl |
| 317 | Me | Me | Me | CH₂═CH₂ | 3-(EtO-C(Me)Pr)-Phenyl |
| 318 | Me | Et | Me | CH₂═CH₂ | 3-(EtO-C(Me)Pr)-Phenyl |
| 317 | (CH₂)₄ | | Me | CH₂═CH₂ | 3-(EtO-C(Me)Pr)-Phenyl |
| 320 | (CH₂)₅ | | Me | CH₂═CH₂ | 3-(EtO-C(Me)Pr)-Phenyl |
| 321 | Me | iPr | Me | O | 3-Br-5-F-Phenyl |
| 322 | Me | Me | Me | Bond | 3-Br-5-F-Phenyl |
| 323 | Me | Et | Me | Bond | 3-Br-5-F-Phenyl |
| 324 | (CH₂)₄ | | Me | Bond | 3-Br-5-F-Phenyl |
| 325 | (CH₂)₅ | | Me | Bond | 3-Br-5-F-Phenyl |
| 326 | Me | Me | Me | O | 3-Br-5-Cl-Phenyl |
| 327 | Me | Et | Me | O | 3-Br-5-Cl-Phenyl |
| 328 | Me | iPr | Me | O | 3-Br-5-Cl-Phenyl |
| 329 | (CH₂)₄ | | Me | O | 3-Br-5-Cl-Phenyl |
| 330 | (CH₂)₅ | | Me | O | 3-Br-5-Cl-Phenyl |
| 331 | Me | Me | Me | S | 3-Br-5-Cl-Phenyl |
| 332 | Me | Me | Me | Bond | 3-Br-5-Cl-Phenyl |
| 333 | Me | Et | Me | Bond | 3-Br-5-Cl-Phenyl |
| 334 | (CH₂)₄ | | Me | Bond | 3-Br-5-Cl-Phenyl |
| 335 | (CH₂)₅ | | Me | Bond | 3-Br-5-Cl-Phenyl |
| 336 | Me | Me | Me | O | 2-F-Phenyl |
| 337 | Me | Et | Me | O | 2-F-Phenyl |
| 338 | (CH₂)₄ | | Me | O | 2-F-Phenyl |
| 339 | (CH₂)₅ | | Me | O | 2-F-Phenyl |
| 340 | Me | Me | Me | S | 2-F-Phenyl |
| 341 | Me | Et | Me | S | 2-F-Phenyl |
| 342 | (CH₂)₄ | | Me | S | 2-F-Phenyl |
| 343 | (CH₂)₅ | | Me | S | 2-F-Phenyl |
| 344 | Me | Me | Me | CH₂CH₂ | 2-F-Phenyl |
| 345 | Me | Et | Me | CH₂CH₂ | 2-F-Phenyl |
| 346 | (CH₂)₄ | | Me | CH₂CH₂ | 2-F-Phenyl |
| 347 | (CH₂)₅ | | Me | CH₂CH₂ | 2-F-Phenyl |
| 348 | Me | Me | Me | CH₂═CH₂ | 2-F-Phenyl |
| 349 | Me | Et | Me | CH₂═CH₂ | 2-F-Phenyl |
| 350 | (CH₂)₄ | | Me | CH₂═CH₂ | 2-F-Phenyl |
| 351 | (CH₂)₅ | | Me | CH₂═CH₂ | 2-F-Phenyl |
| 352 | Me | Me | Me | Bond | 2-F-Phenyl |
| 353 | Me | Et | Me | Bond | 2-F-Phenyl |
| 354 | (CH₂)₄ | | Me | Bond | 2-F-Phenyl |
| 355 | (CH₂)₅ | | Me | Bond | 2-F-Phenyl |
| 356 | Me | Pr | Me | O | 4-Br-3-iPr-Phenyl |
| 357 | Me | Me | Me | Bond | 4-Br-3-iPr-Phenyl |
| 358 | Me | Et | Me | Bond | 4-Br-3-iPr-Phenyl |
| 359 | (CH₂)₄ | | Me | Bond | 4-Br-3-iPr-Phenyl |
| 360 | (CH₂)₅ | | Me | Bond | 4-Br-3-iPr-Phenyl |
| 361 | Me | Me | Me | Bond | 4-Cl-3-CF₃-Phenyl |
| 362 | Me | Et | Me | Bond | 4-Cl-3-CF₃-Phenyl |
| 363 | (CH₂)₄ | | Me | Bond | 4-Cl-3-CF₃-Phenyl |
| 364 | (CH₂)₅ | | Me | Bond | 4-Cl-3-CF₃-Phenyl |
| 365 | Me | Me | Me | O | 4-F-3-CF₃-Phenyl |
| 366 | Me | Et | Me | O | 4-F-3-CF₃-Phenyl |
| 367 | Me | iPr | Me | O | 4-F-3-CF₃-Phenyl |
| 368 | (CH₂)₄ | | Me | O | 4-F-3-CF₃-Phenyl |
| 369 | (CH₂)₄ | | Me | O | 4-F-3-CF₃-Phenyl |
| 370 | Me | Me | Me | O | 5-F-3-CF₃-Phenyl |
| 371 | Me | Et | Me | O | 5-F-3-CF₃-Phenyl |
| 372 | (CH₂)₄ | | Me | O | 5-F-3-CF₃-Phenyl |
| 373 | (CH₂)₅ | | Me | O | 5-F-3-CF₃-Phenyl |
| 374 | Me | Me | Me | O | 4-Cl-3-CF₃-Phenyl |
| 375 | Me | Me | Me | Bond | 4-Cl-3-CF3-Phenyl |
| 376 | Me | Et | Me | Bond | 4-Cl-3-CF3-Phenyl |
| 377 | (CH₂)₄ | | Me | Bond | 4-Cl-3-CF₃-Phenyl |
| 378 | (CH₂)₅ | | Me | Bond | 4-Cl-3-CF₃-Phenyl |
| 379 | Me | Me | Me | O | 4-F-3-CF₃-Phenyl |
| 380 | Me | Et | Me | O | 4-F-3-CF₃-Phenyl |
| 381 | Me | iPr | Me | O | 4-F-3-CF₃-Phenyl |
| 382 | (CH₂)₄ | | Me | O | 4-F-3-CF₃-Phenyl |
| 383 | (CH₂)₄ | | Me | O | 4-F-3-CF₃-Phenyl |
| 384 | Me | Me | Me | O | 5-F-3-CF₃-Phenyl |
| 385 | Me | Et | Me | O | 5-F-3-CF₃-Phenyl |
| 386 | (CH₂)₄ | | Me | O | 5-F-3-CF₃-Phenyl |
| 387 | (CH₂)₅ | | Me | O | 5-F-3-CF₃-Phenyl |
| 388 | Me | Et | Cl | O | 4-Cl-3-tBu-Phenyl |
| 389 | Me | Me | Me | Bond | 4-Cl-3-tBu-Phenyl |
| 390 | Me | Et | Me | Bond | 4-Cl-3-tBu-Phenyl |
| 391 | (CH₂)₄ | | Me | Bond | 4-Cl-3-tBu-Phenyl |
| 392 | (CH₂)₅ | | Me | Bond | 4-Cl-3-tBu-Phenyl |
| 393 | Me | Me | Me | Bond | 4-Cl-2-Me-Phenyl |
| 394 | Me | Et | Me | Bond | 4-Cl-2-Me-Phenyl |
| 395 | (CH₂)₄ | | Me | Bond | 4-Cl-2-Me-Phenyl |
| 396 | (CH₂)₅ | | Me | Bond | 4-Cl-2-Me-Phenyl |
| 397 | Me | Me | Me | Bond | 4-Cl-3-Me-Phenyl |
| 398 | Me | Et | Me | Bond | 4-Cl-3-Me-Phenyl |
| 399 | (CH₂)₄ | | Me | Bond | 4-Cl-3-Me-Phenyl |
| 400 | (CH₂)₅ | | Me | Bond | 4-Cl-3-Me-Phenyl |
| 401 | Me | iPr | Me | O | 4-Cl-3-iPr-Phenyl |
| 402 | Me | cPr | Cl | O | 4-Cl-3-iPr-Phenyl |
| 403 | Me | Me | Me | Bond | 4-Cl-3-iPr-Phenyl |
| 404 | Me | Et | Me | Bond | 4-Cl-3-iPr-Phenyl |
| 405 | (CH₂)₄ | | Me | Bond | 4-Cl-3-iPr-Phenyl |
| 406 | (CH₂)₅ | | Me | Bond | 4-Cl-3-iPr-Phenyl |
| 407 | Me | Et | Me | O | 4-Cl-6-iPr-Pyridin-2-yl |
| 408 | Me | Me | Me | CH₂CH₂ | 4-Cl-6-iPr-Pyridin-2-yl |
| 409 | Me | Et | Me | CH₂CH₂ | 4-Cl-6-iPr-Pyridin-2-yl |
| 410 | (CH₂)₄ | | Me | CH₂CH₂ | 4-Cl-6-iPr-Pyridin-2-yl |
| 411 | (CH₂)₅ | | Me | CH₂CH₂ | 4-Cl-6-iPr-Pyridin-2-yl |
| 412 | Me | Me | Me | Bond | 4-Cl-6-iPr-Pyridin-2-yl |
| 413 | Me | Et | Me | Bond | 4-Cl-6-iPr-Pyridin-2-yl |
| 414 | (CH₂)₄ | | Me | Bond | 4-Cl-6-iPr-Pyridin-2-yl |
| 415 | (CH₂)₅ | | Me | Bond | 4-Cl-6-iPr-Pyridin-2-yl |
| 416 | (CH₂)₅ | | Me | S | 3-(2-Cl-Pyridin-3-yl)-Phenyl |
| 417 | Me | Me | Me | CH₂═CH₂ | 3-(2-Cl-Pyridin-3-yl)-Phenyl |
| 418 | Me | Et | Me | CH₂═CH₂ | 3-(2-Cl-Pyridin-3-yl)-Phenyl |
| 419 | (CH₂)₄ | | Me | CH₂═CH₂ | 3-(2-Cl-Pyridin-3-yl)-Phenyl |
| 420 | (CH₂)₅ | | Me | CH₂═CH₂ | 3-(2-Cl-Pyridin-3-yl)-Phenyl |
| 421 | Me | Me | Me | CH₂CH₂ | 3-(2-Cl-Pyridin-3-yl)-Phenyl |
| 422 | Me | Et | Me | CH₂CH₂ | 3-(2-Cl-Pyridin-3-yl)-Phenyl |
| 423 | (CH₂)₄ | | Me | CH₂CH₂ | 3-(2-Cl-Pyridin-3-yl)-Phenyl |
| 424 | (CH2)5 | | Me | CH₂CH₂ | 3-(2-Cl-Pyridin-3-yl)-Phenyl |
| 425 | Me | Et | Me | Bond | 3-(2-Cl-Pyridin-3-yl)-Phenyl |
| 426 | (CH₂)₄ | | Me | Bond | 3-(2-Cl-Pyridin-3-yl)-Phenyl |
| 427 | (CH₂)₅ | | Me | Bond | 3-(2-Cl-Pyridin-3-yl)-Phenyl |
| 428 | Me | Me | Me | Bond | 3-CF₃-4-Me-Phenyl |
| 429 | Me | Et | Me | Bond | 3-CF₃-4-Me-Phenyl |
| 430 | (CH₂)₄ | | Me | Bond | 3-CF₃-4-Me-Phenyl |
| 431 | (CH₂)₅ | | Me | Bond | 3-CF₃-4-Me-Phenyl |
| 432 | Me | Me | Me | Bond | 4-F-3-OCF₃-Phenyl |
| 433 | Me | Et | Me | Bond | 4-F-3-OCF₃-Phenyl |
| 434 | (CH₂)₄ | | Me | Bond | 4-F-3-OCF₃-Phenyl |
| 435 | (CH₂)₅ | | Me | Bond | 4-F-3-OCF₃-Phenyl |
| 436 | Me | Me | Me | Bond | 3-I-5-CF₃-Phenyl |
| 437 | Me | Et | Me | Bond | 3-I-5-CF₃-Phenyl |
| 438 | (CH₂)₄ | | Me | Bond | 3-I-5-CF₃-Phenyl |
| 439 | (CH₂)₅ | | Me | Bond | 3-I-5-CF₃-Phenyl |
| 440 | Me | iPr | Me | O | 3-tBu-Phenyl |
| 441 | Me | Me | Me | Bond | 3-tBu-Phenyl |
| 442 | Me | Et | Me | Bond | 3-tBu-Phenyl |
| 443 | (CH₂)₄ | | Me | Bond | 3-tBu-Phenyl |
| 444 | (CH₂)₅ | | Me | Bond | 3-tBu-Phenyl |
| 445 | Me | Me | Me | Bond | 4-tBu-Phenyl |
| 446 | Me | Et | Me | Bond | 4-tBu-Phenyl |
| 447 | (CH₂)₄ | | Me | Bond | 4-tBu-Phenyl |
| 448 | (CH₂)₅ | | Me | Bond | 4-tBu-Phenyl |
| 449 | Me | Me | Me | Bond | 4-CN-3-CF₃-Phenyl |
| 450 | Me | Et | Me | Bond | 4-CN-3-CF₃-Phenyl |
| 451 | (CH₂)₄ | | Me | Bond | 4-CN-3-CF₃-Phenyl |
| 452 | (CH₂)₅ | | Me | Bond | 4-CN-3-CF₃-Phenyl |
| 453 | Me | Me | Me | Bond | 4-CF₃-Phenyl |
| 454 | Me | Et | Me | Bond | 4-CF₃-Phenyl |
| 455 | (CH₂)₄ | | Me | Bond | 4-CF₃-Phenyl |
| 456 | (CH₂)₅ | | Me | Bond | 4-CF₃-Phenyl |
| 457 | Me | Me | Me | Bond | 2-Me-Phenyl |
| 458 | Me | Et | Me | Bond | 2-Me-Phenyl |
| 459 | (CH₂)₄ | | Me | Bond | 2-Me-Phenyl |
| 460 | (CH₂)₅ | | Me | Bond | 2-Me-Phenyl |
| 461 | Me | Me | Me | Bond | 4-MeO-Phenyl |
| 462 | Me | Et | Me | Bond | 4-MeO-Phenyl |
| 463 | (CH₂)₄ | | Me | Bond | 4-MeO-Phenyl |
| 464 | (CH₂)₅ | | Me | Bond | 4-MeO-Phenyl |
| 465 | Me | Me | Me | Bond | 3-Ph-O-Phenyl |
| 466 | Me | Et | Me | Bond | 3-Ph-O-Phenyl |
| 467 | (CH₂)₄ | | Me | Bond | 3-Ph-O-Phenyl |
| 468 | (CH₂)₅ | | Me | Bond | 3-Ph-O-Phenyl |
| 469 | Me | Me | Me | Bond | 3-(EtO—C(Me)Pr)-Phenyl |
| 470 | Me | Me | Me | Bond | 3-(EtO—C(Me)Pr)-Phenyl |
| 471 | (CH₂)₄ | | Me | Bond | 3-(EtO—C(Me)Pr)-Phenyl |
| 472 | (CH₂)₅ | | Me | Bond | 3-(EtO—C(Me)Pr)-Phenyl |
| 473 | Me | Me | Me | O | 3-(MeO—C(Me)Pr)-Phenyl |
| 474 | Me | Et | Me | O | 3-(MeO—C(Me)Pr)-Phenyl |
| 475 | (CH₂)₄ | | Me | O | 3-(MeO—C(Me)Pr)-Phenyl |
| 476 | (CH₂)₅ | | Me | O | 3-(MeO—C(Me)Pr)-Phenyl |
| 477 | Me | Me | Me | O | 3-(EtO—C(Me)(H₂C═CH))-Phenyl |
| 478 | Me | Et | Me | O | 3-(EtO—C(Me)(H₂C═CH))-Phenyl |
| 479 | (CH₂)₄ | | Me | O | 3-(EtO—C(Me)(H₂C═CH))-Phenyl |
| 480 | (CH₂)₅ | | Me | O | 3-(EtO—C(Me)(H₂C═CH))-Phenyl |
| 481 | Me | Me | Me | O | 3-(EtO—C(Me)(H₂C═C(Me)CH₂))-Phenyl |
| 482 | Me | Et | Me | O | 3-(EtO—C(Me)(H₂C═C(Me)CH₂))-Phenyl |
| 483 | (CH₂)₄ | | Me | O | 3-(EtO—C(Me)(H₂C═C(Me)CH₂))-Phenyl |
| 484 | (CH₂)₅ | | Me | O | 3-(EtO—C(Me)(H₂C═C(Me)CH₂))-Phenyl |
| 485 | Me | Me | Me | O | 3-(MeO—C(Me)(H₂C═C(Me)CH₂))-Phenyl |
| 486 | Me | Et | Me | O | 3-(MeO—C(Me)(H₂C═C(Me)CH₂))-Phenyl |
| 487 | (CH₂)₄ | | Me | O | 3-(MeO—C(Me)(H₂C═C(Me)CH₂))-Phenyl |
| 488 | (CH₂)₅ | | Me | O | 3-(MeO—C(Me)(H₂C═C(Me)CH₂))-Phenyl |
| 489 | Me | Me | Me | O | 3-(MeO—C(Me)(H₂C═CHCH₂))-Phenyl |
| 490 | Me | Et | Me | O | 3-(MeO—C(Me)(H₂C═CHCH₂))-Phenyl |
| 491 | (CH₂)₄ | | Me | O | 3-(MeO—C(Me)(H₂C═CHCH₂))-Phenyl |
| 492 | (CH₂)₅ | | Me | O | 3-(MeO—C(Me)(H₂C═CHCH₂))-Phenyl |
| 493 | Me | Me | Me | O | 3-(HO—C(Me)(H₂C═CHCH₂))-Phenyl |
| 494 | Me | Et | Me | O | 3-(HO—C(Me)(H₂C═CHCH₂))-Phenyl |
| 495 | (CH₂)₄ | | Me | O | 3-(HO—C(Me)(H₂C═CHCH₂))-Phenyl |
| 496 | (CH₂)₅ | | Me | O | 3-(HO—C(Me)(H₂C═CHCH₂))-Phenyl |
| 497 | Me | Me | Me | O | 3-(EtO—C(Me)(H₂C═CHCH₂))-Phenyl |
| 498 | Me | Et | Me | O | 3-(EtO—C(Me)(H₂C═CHCH₂))-Phenyl |
| 499 | (CH₂)₄ | | Me | O | 3-(EtO—C(Me)(H₂C═CHCH₂))-Phenyl |
| 500 | (CH₂)₅ | | Me | O | 3-(EtO—C(Me)(H₂C═CHCH₂))-Phenyl |
| 501 | Me | Me | Me | O | 3-(MeO—C(Me))Et-Phenyl |
| 502 | Me | Et | Me | O | 3-(MeO—C(Me))Et-Phenyl |
| 503 | (CH₂)₄ | | Me | O | 3-(MeO—C(Me))Et-Phenyl |
| 504 | (CH₂)₅ | | Me | O | 3-(MeO—C(Me))Et-Phenyl |
| 505 | Me | Me | Me | O | 3-(4,5-Dimethyl-1,3-dioxolan-2-yl)-Phenyl |
| 506 | Me | Et | Me | O | 3-(4,5-Dimethyl-1,3-dioxolan-2-yl)-Phenyl |
| 507 | (CH₂)₄ | | Me | O | 3-(4,5-Dimethyl-1,3-dioxolan-2-yl)-Phenyl |
| 508 | (CH₂)₅ | | Me | O | 3-(4,5-Dimethyl-1,3-dioxolan-2-yl)-Phenyl |
| 509 | Me | Me | Me | Bond | 3-(4,5-Dimethyl-1,3-dioxolan-2-yl)-Phenyl |
| 510 | Me | Et | Me | Bond | 3-(4,5-Dimethyl-1,3-dioxolan-2-yl)-Phenyl |
| 511 | (CH₂)₄ | | Me | Bond | 3-(4,5-Dimethyl-1,3-dioxolan-2-yl)-Phenyl |
| 512 | (CH₂)₅ | | Me | Bond | 3-(4,5-Dimethyl-1,3-dioxolan-2-yl)-Phenyl |
| 513 | Me | Me | Me | O | 3-(tBu-carbonyl)-Phenyl |
| 514 | Me | Et | Me | O | 3-(tBu-carbonyl)-Phenyl |
| 515 | (CH₂)₄ | | Me | O | 3-(tBu-carbonyl)-Phenyl |
| 516 | (CH₂)₅ | | Me | O | 3-(tBu-carbonyl)-Phenyl |
| 517 | Me | Me | Me | Bond | 3-(tBu-carbonyl)-Phenyl |
| 518 | Me | Et | Me | Bond | 3-(tBu-carbonyl)-Phenyl |
| 519 | (CH₂)₄ | | Me | Bond | 3-(tBu-carbonyl)-Phenyl |
| 520 | (CH₂)₅ | | Me | Bond | 3-(tBu-carbonyl)-Phenyl |
| 521 | Me | Me | Me | O | 3-(tBu-O-carbonyl)-Phenyl |
| 522 | Me | Et | Me | O | 3-(tBu-O-carbonyl)-Phenyl |
| 523 | (CH₂)₄ | | Me | O | 3-(tBu-O-carbonyl)-Phenyl |
| 524 | (CH₂)₅ | | Me | O | 3-(tBu-O-carbonyl)-Phenyl |
| 525 | Me | Me | Me | Bond | 3-(tBu-O-carbonyl)-Phenyl |
| 526 | Me | Et | Me | Bond | 3-(tBu-O-carbonyl)-Phenyl |
| 527 | (CH₂)₄ | | Me | Bond | 3-(tBu-O-carbonyl)-Phenyl |
| 528 | (CH₂)₅ | | Me | Bond | 3-(tBu-O-carbonyl)-Phenyl |
| 529 | Me | Me | Me | O | 3-(HO—C(Me)(iPr))-Phenyl |
| 530 | Me | Et | Me | O | 3-(HO—C(Me)(iPr))-Phenyl |
| 531 | (CH₂)₄ | | Me | O | 3-(HO—C(Me)(iPr))-Phenyl |
| 532 | (CH₂)₅ | | Me | O | 3-(HO—C(Me)(iPr))-Phenyl |
| 533 | Me | Me | Me | O | 3-(HO—C(Me)(tBu))-Phenyl |
| 534 | Me | Et | Me | O | 3-(HO—C(Me)(tBu))-Phenyl |
| 535 | (CH₂)₄ | | Me | O | 3-(HO—C(Me)(tBu))-Phenyl |
| 536 | (CH₂)₅ | | Me | O | 3-(HO—C(Me)(tBu))-Phenyl |
| 537 | Me | Me | Me | O | 2-Naphthyl |
| 538 | Me | Et | Me | O | 2-Naphthyl |
| 539 | Me | Et | Me | O | 2-Naphthyl |
| 540 | (CH₂)₄ | | Me | O | 2-Naphthyl |
| 541 | (CH₂)₅ | | Me | O | 2-Naphthyl |
| 542 | Me | Et | Me | Bond | 2-Naphthyl |
| 543 | Me | Me | Me | Bond | 2-Naphthyl |
| 544 | (CH₂)₄ | | Me | Bond | 2-Naphthyl |
| 545 | (CH₂)₅ | | Me | Bond | 2-Naphthyl |
| 546 | Me | Me | Me | O | (4-iPr)-thia-3,5-diazol-2-yl |
| 547 | Me | Et | Me | O | (4-iPr)-thia-3,5-diazol-2-yl |
| 548 | (CH₂)₄ | | Me | O | (4-iPr)-thia-3,5-diazol-2-yl |
| 549 | (CH₂)₅ | | Me | O | (4-iPr)-thia-3,5-diazol-2-yl |
| 550 | Me | Me | Me | O | 2,4-Dichlorothiazol-5-yl |
| 551 | Me | Et | Me | O | 2,4-Dichlorothiazol-5-yl |
| 552 | (CH₂)₄ | | Me | O | 2,4-Dichlorothiazol-5-yl |
| 553 | (CH₂)₅ | | Me | O | 2,4-Dichlorothiazol-5-yl |
| 554 | Me | Me | Me | OCH₂ | 2,4-Dichlorothiazol-5-yl |
| 555 | Me | Et | Me | OCH₂ | 2,4-Dichlorothiazol-5-yl |
| 556 | (CH₂)₄ | | Me | OCH₂ | 2,4-Dichlorothiazol-5-yl |
| 557 | (CH₂)₅ | | Me | OCH₂ | 2,4-Dichlorothiazol-5-yl |
| 558 | Me | Me | Me | O | 5-Cl-4-tBu-thiazol-2-yl |
| 559 | Me | Et | Me | O | 5-Cl-4-tBu-thiazol-2-yl |
| 560 | Me | Me | Cl | O | 3-CF₃-4-Cl-Phenyl |
| 561 | Me | Et | Cl | O | 3-CF₃-4-Cl-Phenyl |
| 562 | (CH₂)₄ | | Cl | O | 3-CF₃-4-Cl-Phenyl |
| 563 | (CH₂)₅ | | Cl | O | 3-CF₃-4-Cl-Phenyl |
| 564 | Me | Me | Cl | O | 3-CF₃-5-Cl-Phenyl |
| 565 | Me | Et | Cl | O | 3-CF₃-5-Cl-Phenyl |
| 566 | Me | Pr | Cl | O | 3-CF₃-5-Cl-Phenyl |
| 567 | (CH₂)₄ | | Cl | O | 3-CF₃-5-Cl-Phenyl |
| 568 | (CH₂)₅ | | Cl | O | 3-CF₃-5-Cl-Phenyl |
| 569 | Me | Me | Cl | O | 3-iPr-4-Cl-Phenyl |
| 570 | Me | Et | Cl | O | 3-iPr-4-Cl-Phenyl |
| 571 | Me | Pr | Cl | O | 3-iPr-4-Cl-Phenyl |
| 572 | Me | iPr | Cl | O | 3-iPr-4-Cl-Phenyl |
| 573 | Me | 1-Methyl- | Cl | O | 3-iPr-4-Cl-Phenyl |
| | | propyl | | | |
| 574 | (CH₂)₄ | | Cl | O | 3-iPr-4-Cl-Phenyl |
| 575 | (CH₂)₅ | | Cl | O | 3-iPr-4-Cl-Phenyl |
| 576 | Me | Me | Cl | O | 3-tBu-4-Cl-Phenyl |
| 577 | Me | cPr | Cl | O | 3-tBu-4-Cl-Phenyl |
| 578 | Me | Pr | Cl | O | 3-tBu-4-Cl-Phenyl |
| 579 | Me | 1-Methyl- | Cl | O | 3-tBu-4-Cl-Phenyl |
| | | propyl | | | |
| 580 | (CH₂)₄ | | Cl | O | 3-tBu-4-Cl-Phenyl |
| 581 | (CH₂)₅ | | Cl | O | 3-tBu-4-Cl-Phenyl |
| 582 | Me | Me | Cl | O | 3-Et-4-Cl-Phenyl |
| 583 | Me | Et | Cl | O | 3-Et-4-Cl-Phenyl |
| 584 | Me | Pr | Cl | O | 3-Et-4-Cl-Phenyl |
| 585 | Me | cPr | Cl | O | 3-Et-4-Cl-Phenyl |
| 586 | (CH₂)₄ | | Cl | O | 3-Et-4-Cl-Phenyl |
| 587 | (CH₂)₅ | | Cl | O | 3-Et-4-Cl-Phenyl |
| 588 | Me | Me | Cl | O | 3-iPr-4-Br-Phenyl |
| 589 | Me | Et | Cl | O | 3-iPr-4-Br-Phenyl |
| 590 | Me | Pr | Cl | O | 3-iPr-4-Br-Phenyl |
| 591 | Me | iPr | Cl | O | 3-iPr-4-Br-Phenyl |
| 592 | Me | cPr | Cl | O | 3-iPr-4-Br-Phenyl |
| 593 | (CH₂)₄ | | Cl | O | 3-iPr-4-Br-Phenyl |
| 594 | (CH₂)₅ | | Cl | O | 3-iPr-4-Br-Phenyl |
| 595 | Me | Me | Cl | O | (1-Me-3-tBu)-1,2-pyrazol-5-yl |
| 596 | Me | Et | Cl | O | (1-Me-3-tBu)-1,2-pyrazol-5-yl |
| 597 | Me | Pr | Cl | O | (1-Me-3-tBu)-1,2-pyrazol-5-yl |
| 598 | Me | cPr | Cl | O | (1-Me-3-tBu)-1,2-pyrazol-5-yl |
| 599 | (CH₂)₄ | | Cl | O | (1-Me-3-tBu)-1,2-pyrazol-5-yl |
| 600 | (CH₂)₅ | | Cl | O | (1-Me-3-tBu)-1,2-pyrazol-5-yl |
| 601 | Me | Me | Cl | O | (1-Me-3-iPr)-1,2-pyrazol-5-yl |
| 602 | Me | Et | Cl | O | (1-Me-3-iPr)-1,2-pyrazol-5-yl |
| 603 | Me | Pr | Cl | O | (1-Me-3-iPr)-1,2-pyrazol-5-yl |
| 604 | (CH₂)₄ | | Cl | O | (1-Me-3-iPr)-1,2-pyrazol-5-yl |
| 605 | (CH₂)₅ | | Cl | O | (1-Me-3-iPr)-1,2-pyrazol-5-yl |
| 606 | 2-MeCH(CH₂)₄) | | Cl | O | (1-Me-3-iPr)-1,2-pyrazol-5-yl |
| 607 | Me | Me | Cl | O | (3-tBu)-1,2,4-thiadiazol-5-yl |
| 608 | Me | Et | Cl | O | (3-tBu)-1,2,4-thiadiazol-5-yl |
| 609 | (CH₂)₄ | | Cl | O | (3-tBu)-1,2,4-thiadiazol-5-yl |
| 610 | (CH₂)₅ | | Cl | O | (3-tBu)-1,2,4-thiadiazol-5-yl |
| 611 | Me | Me | Cl | O | 3-(1-Chlorocyclopropyl)-1,2,4- |
| | | | | | thiadiazol-5-yl |
| 612 | Me | Et | Cl | O | 3-(1-Chlorocyclopropyl)-1,2,4- |
| | | | | | thiadiazol-5-yl |
| 613 | (CH₂)₄ | | Cl | O | 3-(1-Chlorocyclopropyl)-1,2,4- |
| | | | | | thiadiazol-5-yl |
| 614 | (CH₂)₅ | | Cl | O | 3-(1-Chlorocyclopropyl)-1,2,4- |
| | | | | | thiadiazol-5-yl |
| 615 | Me | Me | Cl | O | 3-Phenyl-1,2,4-thiadiazol-5-yl |
| 616 | Me | Et | Cl | O | 3-Phenyl-1,2,4-thiadiazol-5-yl |
| 617 | (CH₂)₄ | | Cl | O | 3-Phenyl-1,2,4-thiadiazol-5-yl |
| 618 | (CH₂)₅ | | Cl | O | 3-Phenyl-1,2,4-thiadiazol-5-yl |
| 619 | Me | Me | Cl | O | 4-(4-Chlorophenoxy)-5-fluoropyrimidin- |
| | | | | | 6-yl |
| 620 | Me | Et | Cl | O | 4-(4-Chlorophenoxy)-5-fluoropyrimidin- |
| | | | | | 6-yl |
| 621 | (CH₂)₄ | | Cl | O | 4-(4-Chlorophenoxy)-5-fluoropyrimidin- |
| | | | | | 6-yl |
| 622 | (CH₂)₅ | | Cl | O | 4-(4-Chlorophenoxy)-5-fluoropyrimidin- |
| | | | | | 6-yl |
| 623 | Me | Me | Cl | Bond | 3-CF₃-4-Cl-Phenyl |
| 624 | Me | Et | Cl | Bond | 3-CF₃-4-Cl-Phenyl |
| 625 | (CH₂)₄ | | Cl | Bond | 3-CF₃-4-Cl-Phenyl |
| 626 | (CH₂)₅ | | Cl | Bond | 3-CF₃-4-Cl-Phenyl |
| 627 | Me | Me | Cl | Bond | 3-iPr-4-Cl-Phenyl |
| 628 | Me | Et | Cl | Bond | 3-iPr-4-Cl-Phenyl |
| 629 | (CH₂)₄ | | Cl | Bond | 3-iPr-4-Cl-Phenyl |
| 630 | (CH₂)₅ | | Cl | Bond | 3-iPr-4-Cl-Phenyl |
| 631 | Me | Me | Cl | Bond | 3-CF₃-5-Cl-Phenyl |
| 632 | Me | Et | Cl | Bond | 3-CF₃-5-Cl-Phenyl |
| 633 | (CH₂)₄ | | Cl | Bond | 3-CF₃-5-Cl-Phenyl |
| 634 | (CH₂)₅ | | Cl | Bond | 3-CF₃-5-Cl-Phenyl |
| 635 | Me | Me | F | O | 3-CF₃-4-Cl-Phenyl |
| 636 | Me | Et | F | O | 3-CF₃-4-Cl-Phenyl |
| 637 | (CH₂)₄ | | F | O | 3-CF₃-4-Cl-Phenyl |
| 638 | (CH₂)₅ | | F | O | 3-CF₃-4-Cl-Phenyl |
| 639 | Me | Me | F | O | 3-tBu-4-Cl-Phenyl |
| 640 | Me | Et | F | O | 3-tBu-4-Cl-Phenyl |
| 641 | (CH₂)₄ | | F | O | 3-tBu-4-Cl-Phenyl |
| 642 | (CH₂)₅ | | F | O | 3-tBu-4-Cl-Phenyl |
| 643 | Me | Me | F | O | 3-CF₃-5-Cl-Phenyl |
| 644 | Me | Et | F | O | 3-CF₃-5-Cl-Phenyl |
| 645 | (CH₂)₄ | | F | O | 3-CF₃-5-Cl-Phenyl |
| 646 | (CH₂)₅ | | F | O | 3-CF₃-5-Cl-Phenyl |
| 647 | Me | Me | F | O | (3-tBu)-1,2,4-thiadiazol-5-yl |
| 648 | Me | Et | F | O | (3-tBu)-1,2,4-thiadiazol-5-yl |
| 649 | (CH₂)₄ | | F | O | (3-tBu)-1,2,4-thiadiazol-5-yl |
| 650 | (CH₂)₅ | | F | O | (3-tBu)-1,2,4-thiadiazol-5-yl |
| 651 | Me | Me | F | O | 3-Phenyl-1,2,4-thiadiazol-5-yl |
| 652 | Me | Et | F | O | 3-Phenyl-1,2,4-thiadiazol-5-yl |
| 653 | (CH₂)₄ | | F | O | 3-Phenyl-1,2,4-thiadiazol-5-yl |
| 654 | (CH₂)₅ | | F | O | 3-Phenyl-1,2,4-thiadiazol-5-yl |
| 655 | Me | Me | F | Bond | 3-CF₃-4-Cl-Phenyl |
| 656 | Me | Et | F | Bond | 3-CF₃-4-Cl-Phenyl |
| 657 | (CH₂)₄ | | F | Bond | 3-CF₃-4-Cl-Phenyl |
| 658 | (CH₂)₅ | | F | Bond | 3-CF₃-4-Cl-Phenyl |
| 659 | Me | Me | F | Bond | 3-CF₃-5-Cl-Phenyl |
| 660 | Me | Et | F | Bond | 3-CF₃-5-Cl-Phenyl |
| 661 | (CH₂)₄ | | F | Bond | 3-CF₃-5-Cl-Phenyl |
| 662 | (CH₂)₅ | | F | Bond | 3-CF₃-5-Cl-Phenyl |
| 663 | Me | Me | F | Bond | 4-Chlorophenyl |
| 664 | Me | Et | F | Bond | 4-Chlorophenyl |
| 665 | Me | iPr | F | Bond | 4-Chlorophenyl |
| 666 | (CH₂)₄ | | F | Bond | 4-Chlorophenyl |
| 667 | (CH₂)₅ | | F | Bond | 4-Chlorophenyl |
| 668 | Me | Me | F | Bond | 4-tBuphenyl |
| 669 | Me | Et | F | Bond | 4-tBuphenyl |
| 670 | (CH₂)₄ | | F | Bond | 4-tBuphenyl |
| 671 | (CH₂)₅ | | F | Bond | 4-tBuphenyl |
| 672 | Me | Me | F | Bond | 4-tButoxyphenyl |
| 673 | Me | Et | F | Bond | 4-tButoxyphenyl |
| 674 | (CH₂)₄ | | F | Bond | 4-tButoxyphenyl |
| 675 | (CH₂)₅ | | F | Bond | 4-tButoxyphenyl |
| 676 | Me | Me | F | Bond | 3-tBuphenyl |
| 677 | Me | Et | F | Bond | 3-tBuphenyl |
| 678 | (CH₂)₄ | | F | Bond | 3-tBuphenyl |
| 679 | (CH₂)₅ | | F | Bond | 3-tBuphenyl |
| 680 | Me | Me | F | Bond | 2-(Cyclopropyl)-1,3,4-thiadiazol-5-yl |
| 681 | Me | Et | F | Bond | 2-(Cyclopropyl)-1,3,4-thiadiazol-5-yl |
| 682 | (CH₂)₄ | | F | Bond | 2-(Cyclopropyl)-1,3,4-thiadiazol-5-yl |
| 683 | (CH₂)₅ | | F | Bond | 2-(Cyclopropyl)-1,3,4-thiadiazol-5-yl |
| 684 | Me | Me | Br | O | 3-CF₃-4-Cl-Phenyl |
| 685 | Me | Et | Br | O | 3-CF₃-4-Cl-Phenyl |
| 686 | (CH₂)₄ | | Br | O | 3-CF₃-4-Cl-Phenyl |
| 687 | (CH₂)₅ | | Br | O | 3-CF₃-4-Cl-Phenyl |
| 688 | Me | Me | Br | O | 3-CF₃-5-Cl-Phenyl |
| 689 | Me | Et | Br | O | 3-CF₃-5-Cl-Phenyl |
| 690 | (CH₂)₄ | | Br | O | 3-CF₃-5-Cl-Phenyl |
| 691 | (CH₂)₅ | | Br | O | 3-CF₃-5-Cl-Phenyl |
| 692 | Me | Me | Br | O | 3-tBu-Phenyl |
| 693 | Me | Et | Br | O | 3-tBu-Phenyl |
| 694 | (CH₂)₄ | | Br | O | 3-tBu-Phenyl |
| 695 | (CH₂)₅ | | Br | O | 3-tBu-Phenyl |
| 696 | Me | Me | Br | O | 3-tBu-4-Cl-Phenyl |
| 697 | Me | Et | Br | O | 3-tBu-4-Cl-Phenyl |
| 698 | (CH₂)₄ | | Br | O | 3-tBu-4-Cl-Phenyl |
| 699 | (CH₂)₅ | | Br | O | 3-tBu-4-Cl-Phenyl |
| 700 | Me | Me | Br | O | 3-iPr-4-Cl-Phenyl |
| 701 | Me | Et | Br | O | 3-iPr-4-Cl-Phenyl |
| 702 | (CH₂)₄ | | Br | O | 3-iPr-4-Cl-Phenyl |
| 703 | (CH₂)₅ | | Br | O | 3-iPr-4-Cl-Phenyl |
| 704 | Me | Me | Br | O | 2-(Cyclopropyl)-1,3,4-thiadiazol-5-yl |
| 705 | Me | Et | Br | O | 2-(Cyclopropyl)-1,3,4-thiadiazol-5-yl |
| 706 | (CH₂)₄ | | Br | O | 2-(Cyclopropyl)-1,3,4-thiadiazol-5-yl |
| 707 | (CH₂)₅ | | Br | O | 2-(Cyclopropyl)-1,3,4-thiadiazol-5-yl |
| 708 | Me | Me | Br | Bond | 3-CF₃-4-Cl-Phenyl |
| 709 | Me | Et | Br | Bond | 3-CF₃-4-Cl-Phenyl |
| 710 | (CH₂)₄ | | Br | Bond | 3-CF₃-4-Cl-Phenyl |
| 711 | (CH₂)₅ | | Br | Bond | 3-CF₃-4-Cl-Phenyl |
| 712 | Me | Me | Br | Bond | 3-CF₃-5-Cl-Phenyl |
| 713 | Me | Et | Br | Bond | 3-CF₃-5-Cl-Phenyl |
| 714 | (CH₂)₄ | | Br | Bond | 3-CF₃-5-Cl-Phenyl |
| 715 | (CH₂)₅ | | Br | Bond | 3-CF₃-5-Cl-Phenyl |
| 716 | Me | Me | Br | Bond | 3-tBu-Phenyl |
| 717 | Me | Et | Br | Bond | 3-tBu-Phenyl |
| 718 | (CH₂)₄ | | Br | Bond | 3-tBu-Phenyl |
| 719 | (CH₂)₅ | | Br | Bond | 3-tBu-Phenyl |
| 720 | Me | Me | OMe | O | 3-CF₃-4-Cl-Phenyl |
| 721 | Me | Et | OMe | O | 3-CF₃-4-Cl-Phenyl |
| 722 | (CH₂)₄ | | OMe | O | 3-CF₃-4-Cl-Phenyl |
| 723 | (CH₂)₅ | | OMe | O | 3-CF₃-4-Cl-Phenyl |
| 724 | Me | Me | OMe | O | 3-CF₃-5-Cl-Phenyl |
| 725 | Me | Et | OMe | O | 3-CF₃-5-Cl-Phenyl |
| 726 | (CH₂)₄ | | OMe | O | 3-CF₃-5-Cl-Phenyl |
| 727 | (CH₂)₅ | | OMe | O | 3-CF₃-5-Cl-Phenyl |
| 728 | Me | Me | OMe | O | 3-iPr-4-Cl-Phenyl |
| 729 | Me | Et | OMe | O | 3-iPr-4-Cl-Phenyl |
| 730 | (CH₂)₄ | | OMe | O | 3-iPr-4-Cl-Phenyl |
| 731 | (CH₂)₅ | | OMe | O | 3-iPr-4-Cl-Phenyl |
| 732 | Me | Me | OMe | O | 3-tBu-4-Cl-Phenyl |
| 733 | Me | Et | OMe | O | 3-tBu-4-Cl-Phenyl |
| 734 | (CH₂)₄ | | OMe | O | 3-tBu-4-Cl-Phenyl |
| 735 | (CH₂)₅ | | OMe | O | 3-tBu-4-Cl-Phenyl |
| 736 | Me | Me | OMe | O | 2-(Cyclopropyl)-1,3,4-thiadiazol-5-yl |
| 737 | Me | Et | OMe | O | 2-(Cyclopropyl)-1,3,4-thiadiazol-5-yl |
| 738 | (CH₂)₄ | | OMe | O | 2-(Cyclopropyl)-1,3,4-thiadiazol-5-yl |
| 739 | (CH₂)₅ | | OMe | O | 2-(Cyclopropyl)-1,3,4-thiadiazol-5-yl |
| 740 | Me | Me | OMe | Bond | 3-CF₃-4-Cl-Phenyl |
| 741 | Me | Et | OMe | Bond | 3-CF₃-4-Cl-Phenyl |
| 742 | (CH₂)₄ | | OMe | Bond | 3-CF₃-4-Cl-Phenyl |
| 743 | (CH₂)₅ | | OMe | Bond | 3-CF₃-4-Cl-Phenyl |
| 744 | Me | Me | OMe | Bond | 3-CF₃-5-Cl-Phenyl |
| 745 | Me | Et | OMe | Bond | 3-CF₃-5-Cl-Phenyl |
| 746 | (CH₂)₄ | | OMe | Bond | 3-CF₃-5-Cl-Phenyl |
| 747 | (CH₂)₅ | | OMe | Bond | 3-CF₃-5-Cl-Phenyl |
| 748 | Me | Me | OMe | Bond | 2-(Cyclopropyl)-1,3,4-thiadiazol-5-yl |
| 749 | Me | Et | OMe | Bond | 2-(Cyclopropyl)-1,3,4-thiadiazol-5-yl |
| 750 | (CH₂)₄ | | OMe | Bond | 2-(Cyclopropyl)-1,3,4-thiadiazol-5-yl |
| 751 | (CH₂)₅ | | OMe | Bond | 2-(Cyclopropyl)-1,3,4-thiadiazol-5-yl |
| 752 | Me | Me | CF₃ | O | 3-CF₃-4-Cl-Phenyl |
| 753 | Me | Et | CF₃ | O | 3-CF₃-4-Cl-Phenyl |
| 754 | (CH₂)₄ | | CF₃ | O | 3-CF₃-4-Cl-Phenyl |
| 755 | (CH₂)₅ | | CF₃ | O | 3-CF₃-4-Cl-Phenyl |
| 756 | Me | Me | CF₃ | O | 3-CF₃-5-Cl-Phenyl |
| 757 | Me | Et | CF₃ | O | 3-CF₃-5-Cl-Phenyl |
| 758 | (CH₂)₄ | | CF₃ | O | 3-CF₃-5-Cl-Phenyl |
| 759 | (CH₂)₅ | | CF₃ | O | 3-CF₃-5-Cl-Phenyl |
| 760 | Me | Me | CF₃ | O | 3-tBu-Phenyl |
| 761 | Me | Et | CF₃ | O | 3-tBu-Phenyl |
| 762 | (CH₂)₄ | | CF₃ | O | 3-tBu-Phenyl |
| 763 | (CH₂)₅ | | CF₃ | O | 3-tBu-Phenyl |
| 764 | Me | Me | CF₃ | Bond | 3-CF₃-4-Cl-Phenyl |
| 765 | Me | Et | CF₃ | Bond | 3-CF₃-4-Cl-Phenyl |
| 766 | (CH₂)₄ | | CF₃ | Bond | 3-CF₃-4-Cl-Phenyl |
| 767 | (CH₂)₅ | | CF₃ | Bond | 3-CF₃-4-Cl-Phenyl |
| 768 | Me | Me | CF₃ | Bond | 3-CF₃-5-Cl-Phenyl |
| 769 | Me | Et | CF₃ | Bond | 3-CF₃-5-Cl-Phenyl |
| 770 | (CH₂)₄ | | CF₃ | Bond | 3-CF₃-5-Cl-Phenyl |
| 771 | (CH₂)₅ | | CF₃ | Bond | 3-CF₃-5-Cl-Phenyl |
| 772 | Me | Me | CF₃ | Bond | 4-tBu-Phenyl |
| 773 | Me | Et | CF₃ | Bond | 4-tBu-Phenyl |
| 774 | (CH₂)₄ | | CF₃ | Bond | 4-tBu-Phenyl |
| 775 | (CH₂)₅ | | CF₃ | Bond | 4-tBu-Phenyl |
| 776 | Me | Me | CF₂H | O | 3-CF₃-4-Cl-Phenyl |
| 777 | Me | Et | CF₂H | O | 3-CF₃-4-Cl-Phenyl |
| 778 | (CH₂)₄ | | CF₂H | O | 3-CF₃-4-Cl-Phenyl |
| 779 | (CH₂)₅ | | CF₂H | O | 3-CF₃-4-Cl-Phenyl |
| 780 | Me | Me | CF₂H | O | 3-CF₃-5-Cl-Phenyl |
| 781 | Me | Et | CF₂H | O | 3-CF₃-5-Cl-Phenyl |
| 782 | (CH₂)₄ | | CF₂H | O | 3-CF₃-5-Cl-Phenyl |
| 783 | (CH₂)₅ | | CF₂H | O | 3-CF₃-5-Cl-Phenyl |
| 784 | Me | Me | CF₂H | O | 3-iPr-Phenyl |
| 785 | Me | Et | CF₂H | O | 3-iPr-Phenyl |
| 786 | (CH₂)₄ | | CF₂H | O | 3-iPr-Phenyl |
| 787 | (CH₂)₅ | | CF₂H | O | 3-iPr-Phenyl |
| 788 | Me | Me | CF₂H | Bond | 3-CF₃-4-Cl-Phenyl |
| 789 | Me | Et | CF₂H | Bond | 3-CF₃-4-Cl-Phenyl |
| 790 | (CH₂)₄ | | CF₂H | Bond | 3-CF₃-4-Cl-Phenyl |
| 791 | (CH₂)₅ | | CF₂H | Bond | 3-CF₃-4-Cl-Phenyl |
| 792 | Me | Me | CF₂H | Bond | 3-CF₃-5-Cl-Phenyl |
| 793 | Me | Et | CF₂H | Bond | 3-CF₃-5-Cl-Phenyl |
| 794 | (CH₂)₄ | | CF₂H | Bond | 3-CF₃-5-Cl-Phenyl |
| 795 | (CH₂)₅ | | CF₂H | Bond | 3-CF₃-5-Cl-Phenyl |
| 796 | Me | Me | CF₂H | Bond | 3-iPr-Phenyl |
| 797 | Me | Et | CF₂H | Bond | 3-iPr-Phenyl |

| | | | | |
|---|---|---|---|---|
| 798 | (CH₂)₄ | CF₂H | Bond | 3-iPr-Phenyl |
| 799 | (CH₂)₅ | CF₂H | Bond | 3-iPr-Phenyl |

TABLE 2
Compounds according to the invention of formula (I) (in table 2, R⁴ is not methyl)

| | |
|---|---|
| | (I) |
| 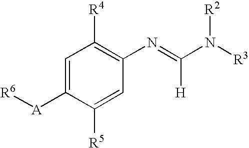 | |

| No. | R² | R³ | R⁴ | R⁵ | A | R⁶ |
|---|---|---|---|---|---|---|
| 800 | Me | Me | Cl | Cl | O | Phenyl |
| 801 | Me | Et | Cl | Cl | O | Phenyl |
| 802 | Me | iPr | Cl | Cl | O | Phenyl |
| 803 | (CH₂)₄ | | Cl | Cl | O | Phenyl |
| 804 | (CH₂)₅ | | Cl | Cl | O | Phenyl |
| 805 | Me | Me | Cl | Cl | O | 3-tBu-Phenyl |
| 806 | Me | Et | Cl | Cl | O | 3-tBu-Phenyl |
| 807 | Me | iPr | Cl | Cl | O | 3-tBu-Phenyl |
| 808 | (CH₂)₄ | | Cl | Cl | O | 3-tBu-Phenyl |
| 809 | (CH₂)₅ | | Cl | Cl | O | 3-tBu-Phenyl |
| 810 | Me | Me | Cl | Cl | O | 3-tBu-4-Cl-Phenyl |
| 811 | Me | Et | Cl | Cl | O | 3-tBu-4-Cl-Phenyl |
| 812 | Me | iPr | Cl | Cl | O | 3-tBu-4-Cl-Phenyl |
| 813 | (CH₂)₄ | | Cl | Cl | O | 3-tBu-4-Cl-Phenyl |
| 814 | (CH₂)₅ | | Cl | Cl | O | 3-tBu-4-Cl-Phenyl |
| 815 | Me | Me | Cl | Cl | O | 3-CF₃-4-Cl-Phenyl |
| 816 | Me | Et | Cl | Cl | O | 3-CF₃-4-Cl-Phenyl |
| 817 | Me | iPr | Cl | Cl | O | 3-CF₃-4-Cl-Phenyl |
| 818 | (CH₂)₄ | | Cl | Cl | O | 3-CF₃-4-Cl-Phenyl |
| 819 | (CH₂)₅ | | Cl | Cl | O | 3-CF₃-4-Cl-Phenyl |
| 820 | Me | Me | Cl | Cl | O | 3-CF₃-5-Cl-Phenyl |
| 821 | Me | Et | Cl | Cl | O | 3-CF₃-5-Cl-Phenyl |
| 822 | (CH₂)₄ | | Cl | Cl | O | 3-CF₃-5-Cl-Phenyl |
| 823 | (CH₂)₅ | | Cl | Cl | O | 3-CF₃-5-Cl-Phenyl |
| 824 | Me | Me | Cl | Cl | O | (3-tBu)-1,2,4-thiadiazol-5-yl |
| 825 | Me | Et | Cl | Cl | O | (3-tBu)-1,2,4-thiadiazol-5-yl |
| 826 | Me | iPr | Cl | Cl | O | (3-tBu)-1,2,4-thiadiazol-5-yl |
| 827 | (CH₂)₄ | | Cl | Cl | O | (3-tBu)-1,2,4-thiadiazol-5-yl |
| 828 | (CH₂)₅ | | Cl | Cl | O | (3-tBu)-1,2,4-thiadiazol-5-yl |
| 829 | Me | Me | F | F | O | 3-tBu-Phenyl |
| 830 | Me | Et | F | F | O | 3-tBu-Phenyl |
| 831 | (CH₂)₄ | | F | F | O | 3-tBu-Phenyl |
| 832 | (CH₂)₅ | | F | F | O | 3-tBu-Phenyl |
| 833 | Me | Me | F | F | O | 3-tBu-4-Cl-Phenyl |
| 834 | Me | Et | F | F | O | 3-tBu-4-Cl-Phenyl |
| 835 | (CH₂)₄ | | F | F | O | 3-tBu-4-Cl-Phenyl |
| 836 | (CH₂)₅ | | F | F | O | 3-tBu-4-Cl-Phenyl |
| 837 | Me | Me | F | F | O | 3-CF₃-4-Cl-Phenyl |
| 838 | Me | Et | F | F | O | 3-CF₃-4-Cl-Phenyl |
| 839 | (CH₂)₄ | | F | F | O | 3-CF₃-4-Cl-Phenyl |
| 840 | (CH₂)₅ | | F | F | O | 3-CF₃-4-Cl-Phenyl |
| 841 | Me | Me | F | F | O | 3-CF₃-5-Cl-Phenyl |
| 842 | Me | Et | F | F | O | 3-CF₃-5-Cl-Phenyl |
| 843 | (CH₂)₄ | | F | F | O | 3-CF₃-5-Cl-Phenyl |
| 844 | (CH₂)₅ | | F | F | O | 3-CF₃-5-Cl-Phenyl |
| 845 | Me | Me | F | F | Bond | 4-tBu-Phenyl |
| 846 | Me | Et | F | F | Bond | 4-tBu-Phenyl |
| 847 | (CH₂)₄ | | F | F | Bond | 4-tBu-Phenyl |
| 848 | (CH₂)₅ | | F | F | Bond | 4-tBu-Phenyl |
| 849 | Me | Me | F | F | Bond | 3-tBu-4-Cl-Phenyl |
| 850 | Me | Et | F | F | Bond | 3-tBu-4-Cl-Phenyl |
| 851 | (CH₂)₄ | | F | F | Bond | 3-tBu-4-Cl-Phenyl |
| 852 | (CH₂)₅ | | F | F | Bond | 3-tBu-4-Cl-Phenyl |
| 853 | Me | Me | F | F | Bond | 3-CF₃-4-Cl-Phenyl |
| 854 | Me | Et | F | F | Bond | 3-CF₃-4-Cl-Phenyl |
| 855 | (CH₂)₄ | | F | F | Bond | 3-CF₃-4-Cl-Phenyl |
| 856 | (CH₂)₅ | | F | F | Bond | 3-CF₃-4-Cl-Phenyl |
| 857 | Me | Me | F | F | Bond | 3-CF₃-5-Cl-Phenyl |
| 858 | Me | Et | F | F | Bond | 3-CF₃-5-Cl-Phenyl |
| 859 | (CH₂)₄ | | F | F | Bond | 3-CF₃-5-Cl-Phenyl |
| 860 | (CH₂)₅ | | F | F | Bond | 3-CF₃-5-Cl-Phenyl |
| 861 | Me | Me | I | Cl | O | 3-tBu-Phenyl |
| 862 | Me | Et | I | Cl | O | 3-tBu-Phenyl |
| 863 | (CH₂)₄ | | I | Cl | O | 3-tBu-Phenyl |
| 864 | (CH₂)₅ | | I | Cl | O | 3-tBu-Phenyl |
| 865 | Me | Me | I | Cl | O | 3-CF₃-4-Cl-Phenyl |
| 866 | Me | Et | I | Cl | O | 3-CF₃-4-Cl-Phenyl |
| 867 | (CH₂)₄ | | I | Cl | O | 3-CF₃-4-Cl-Phenyl |
| 868 | (CH₂)₅ | | I | Cl | O | 3-CF₃-4-Cl-Phenyl |
| 869 | Me | Me | Cl | Me | O | 3-tBu-Phenyl |
| 870 | Me | Et | Cl | Me | O | 3-tBu-Phenyl |
| 871 | Me | iPr | Cl | Me | O | 3-tBu-Phenyl |
| 872 | (CH₂)₄ | | Cl | Me | O | 3-tBu-Phenyl |
| 873 | (CH₂)₅ | | Cl | Me | O | 3-tBu-Phenyl |
| 874 | Me | Me | Cl | Me | O | 3-iPr-Phenyl |
| 875 | Me | Et | Cl | Me | O | 3-iPr-Phenyl |
| 876 | Me | iPr | Cl | Me | O | 3-iPr-Phenyl |
| 877 | (CH₂)₄ | | Cl | Me | O | 3-iPr-Phenyl |
| 878 | (CH₂)₅ | | Cl | Me | O | 3-iPr-Phenyl |
| 879 | Me | Me | Cl | Me | O | 3-OPh |
| 880 | Me | Et | Cl | Me | O | 3-OPh |
| 881 | (CH₂)₄ | | Cl | Me | O | 3-OPh |
| 882 | (CH₂)₅ | | Cl | Me | O | 3-OPh |
| 883 | Me | Me | Cl | Me | O | 4-tBu-Phenyl |
| 884 | Me | Et | Cl | Me | O | 4-tBu-Phenyl |
| 885 | (CH₂)₄ | | Cl | Me | O | 4-tBu-Phenyl |
| 886 | (CH₂)₅ | | Cl | Me | O | 4-tBu-Phenyl |
| 887 | Me | Me | Cl | Me | O | 4-iPr-Phenyl |
| 888 | Me | Et | Cl | Me | O | 4-iPr-Phenyl |
| 889 | Me | iPr | Cl | Me | O | 4-iPr-Phenyl |
| 890 | (CH₂)₄ | | Cl | Me | O | 4-iPr-Phenyl |
| 891 | (CH₂)₅ | | Cl | Me | O | 4-iPr-Phenyl |
| 892 | Me | Me | Cl | Me | O | 3-CF₃-4-Cl-Phenyl |
| 893 | Me | Et | Cl | Me | O | 3-CF₃-4-Cl-Phenyl |
| 894 | Me | iPr | Cl | Me | O | 3-CF₃-4-Cl-Phenyl |
| 895 | (CH₂)₄ | | Cl | Me | O | 3-CF₃-4-Cl-Phenyl |
| 896 | (CH₂)₅ | | Cl | Me | O | 3-CF₃-4-Cl-Phenyl |
| 897 | Me | Me | Cl | Me | O | 3-iPr-4-Cl-Phenyl |
| 898 | Me | Et | Cl | Me | O | 3-iPr-4-Cl-Phenyl |
| 899 | Me | iPr | Cl | Me | O | 3-iPr-4-Cl-Phenyl |
| 900 | (CH₂)₄ | | Cl | Me | O | 3-iPr-4-Cl-Phenyl |
| 901 | (CH₂)₅ | | Cl | Me | O | 3-iPr-4-Cl-Phenyl |
| 902 | Me | Me | Cl | Me | O | 3-tBu-4-Cl-Phenyl |
| 903 | Me | Et | Cl | Me | O | 3-tBu-4-Cl-Phenyl |
| 904 | Me | iPr | Cl | Me | O | 3-tBu-4-Cl-Phenyl |
| 905 | (CH₂)₄ | | Cl | Me | O | 3-tBu-4-Cl-Phenyl |
| 906 | (CH₂)₅ | | Cl | Me | O | 3-tBu-4-Cl-Phenyl |
| 907 | Me | Me | Cl | Me | O | 3-CF₃-5-Cl-Phenyl |
| 908 | Me | Et | Cl | Me | O | 3-CF₃-5-Cl-Phenyl |
| 909 | (CH₂)₄ | | Cl | Me | O | 3-CF₃-5-Cl-Phenyl |
| 910 | (CH₂)₅ | | Cl | Me | O | 3-CF₃-5-Cl-Phenyl |
| 911 | Me | Me | Cl | Me | O | (4-(4-Chlorophenyl)-5-fluoropyrimidin-5-yl) |
| 912 | Me | Et | Cl | Me | O | (4-(4-Chlorophenyl)-5-fluoropyrimidin-5-yl) |
| 913 | (CH₂)₄ | | Cl | Me | O | (4-(4-Chlorophenyl)-5-fluoropyrimidin-5-yl) |
| 914 | (CH₂)₅ | | Cl | Me | O | (4-(4-Chlorophenyl)-5-fluoropyrimidin-5-yl) |
| 915 | Me | Me | Cl | Me | O | (3-tBu)-1,2,4-thiadiazol-5-yl |
| 916 | Me | Et | Cl | Me | O | (3-tBu)-1,2,4-thiadiazol-5-yl |
| 917 | (CH₂)₄ | | Cl | Me | O | (3-tBu)-1,2,4-thiadiazol-5-yl |
| 918 | (CH₂)₅ | | Cl | Me | O | (3-tBu)-1,2,4-thiadiazol-5-yl |
| 919 | Me | Me | Cl | Me | Bond | 3-iPr-Phenyl |
| 920 | Me | Et | Cl | Me | Bond | 3-iPr-Phenyl |
| 921 | (CH₂)₄ | | Cl | Me | Bond | 3-iPr-Phenyl |
| 922 | (CH₂)₅ | | Cl | Me | Bond | 3-iPr-Phenyl |
| 923 | Me | Me | Cl | Me | Bond | 3-tBu-4-Cl-Phenyl |
| 924 | Me | Et | Cl | Me | Bond | 3-tBu-4-Cl-Phenyl |
| 925 | (CH₂)₄ | | Cl | Me | Bond | 3-tBu-4-Cl-Phenyl |
| 926 | (CH₂)₅ | | Cl | Me | Bond | 3-tBu-4-Cl-Phenyl |
| 927 | Me | Me | Cl | Me | Bond | 3-CF₃-4-Cl-Phenyl |
| 928 | Me | Et | Cl | Me | Bond | 3-CF₃-4-Cl-Phenyl |
| 929 | (CH₂)₄ | | Cl | Me | Bond | 3-CF₃-4-Cl-Phenyl |
| 930 | (CH₂)₅ | | Cl | Me | Bond | 3-CF₃-4-Cl-Phenyl |
| 931 | Me | Me | Cl | Me | Bond | 3-CF₃-5-Cl-Phenyl |
| 932 | Me | Et | Cl | Me | Bond | 3-CF₃-5-Cl-Phenyl |
| 933 | (CH₂)₄ | | Cl | Me | Bond | 3-CF₃-5-Cl-Phenyl |
| 934 | (CH₂)₅ | | Cl | Me | Bond | 3-CF₃-5-Cl-Phenyl |
| 935 | Me | Me | Cl | Me | Bond | 4-tBu-Phenyl |
| 936 | Me | Et | Cl | Me | Bond | 4-tBu-Phenyl |
| 937 | (CH₂)₄ | | Cl | Me | Bond | 4-tBu-Phenyl |
| 938 | (CH₂)₅ | | Cl | Me | Bond | 4-tBu-Phenyl |
| 939 | Me | Me | Cl | CF₃ | O | 3-tBu-Phenyl |
| 940 | Me | Et | Cl | CF₃ | O | 3-tBu-Phenyl |
| 941 | (CH₂)₄ | | Cl | CF₃ | O | 3-tBu-Phenyl |
| 942 | (CH₂)₅ | | Cl | CF₃ | O | 3-tBu-Phenyl |
| 943 | Me | Me | Cl | CF₃ | O | 3-tBu-4-Cl-Phenyl |
| 944 | Me | Et | Cl | CF₃ | O | 3-tBu-4-Cl-Phenyl |
| 945 | (CH₂)₄ | | Cl | CF₃ | O | 3-tBu-4-Cl-Phenyl |
| 946 | (CH₂)₅ | | Cl | CF₃ | O | 3-tBu-4-Cl-Phenyl |
| 947 | Me | Me | Cl | CF₃ | O | 3-CF₃-4-Cl-Phenyl |
| 948 | Me | Et | Cl | CF₃ | O | 3-CF₃-4-Cl-Phenyl |
| 949 | Me | iPr | Cl | CF₃ | O | 3-CF₃-4-Cl-Phenyl |
| 950 | (CH₂)₄ | | Cl | CF₃ | O | 3-CF₃-4-Cl-Phenyl |
| 951 | (CH₂)₅ | | Cl | CF₃ | O | 3-CF₃-4-Cl-Phenyl |
| 952 | Me | Me | Cl | CF₃ | O | 3-CF₃-5-Cl-Phenyl |
| 953 | Me | Et | Cl | CF₃ | O | 3-CF₃-5-Cl-Phenyl |
| 954 | (CH₂)₄ | | Cl | CF₃ | O | 3-CF₃-5-Cl-Phenyl |
| 955 | (CH₂)₅ | | Cl | CF₃ | O | 3-CF₃-5-Cl-Phenyl |
| 956 | Me | Me | Cl | CF₃ | O | 3-Phenyl |
| 957 | Me | Et | Cl | CF₃ | O | 3-Phenyl |
| 958 | Me | iPr | Cl | CF₃ | O | 3-Phenyl |
| 959 | (CH₂)₄ | | Cl | CF₃ | O | 3-Phenyl |
| 960 | (CH₂)₅ | | Cl | CF₃ | O | 3-Phenyl |
| 961 | Me | Me | Cl | CF₃ | Bond | 4-tBu-Phenyl |
| 962 | Me | Et | Cl | CF₃ | Bond | 4-tBu-Phenyl |
| 963 | (CH₂)₄ | | Cl | CF₃ | Bond | 4-tBu-Phenyl |
| 964 | (CH₂)₅ | | Cl | CF₃ | Bond | 4-tBu-Phenyl |
| 965 | Me | Me | Cl | CF₃ | Bond | 3-tBu-4-Cl-Phenyl |
| 966 | Me | Et | Cl | CF₃ | Bond | 3-tBu-4-Cl-Phenyl |
| 967 | (CH₂)₄ | | Cl | CF₃ | Bond | 3-tBu-4-Cl-Phenyl |
| 968 | (CH₂)₅ | | Cl | CF₃ | Bond | 3-tBu-4-Cl-Phenyl |
| 969 | Me | Me | Cl | CF₃ | Bond | 3-CF₃-4-Cl-Phenyl |
| 970 | Me | Et | Cl | CF₃ | Bond | 3-CF₃-4-Cl-Phenyl |
| 971 | (CH₂)₄ | | Cl | CF₃ | Bond | 3-CF₃-4-Cl-Phenyl |
| 972 | (CH₂)₅ | | Cl | CF₃ | Bond | 3-CF₃-4-Cl-Phenyl |
| 973 | Me | Me | Cl | CF₃ | Bond | 3-tBu-Phenyl |
| 974 | Me | Et | Cl | CF₃ | Bond | 3-tBu-Phenyl |
| 975 | (CH₂)₄ | | Cl | CF₃ | Bond | 3-tBu-Phenyl |
| 976 | (CH₂)₅ | | Cl | CF₃ | Bond | 3-tBu-Phenyl |
| 977 | Me | Me | OMe | OMe | O | 3-tBu-Phenyl |
| 978 | Me | Et | OMe | OMe | O | 3-tBu-Phenyl |
| 979 | (CH₂)₄ | | OMe | OMe | O | 3-tBu-Phenyl |
| 980 | (CH₂)₅ | | OMe | OMe | O | 3-tBu-Phenyl |
| 981 | Me | Me | OMe | OMe | O | 3-tBu-4-Cl-Phenyl |
| 982 | Me | Et | OMe | OMe | O | 3-tBu-4-Cl-Phenyl |
| 983 | (CH₂)₄ | | OMe | OMe | O | 3-tBu-4-Cl-Phenyl |
| 984 | (CH₂)₅ | | OMe | OMe | O | 3-tBu-4-Cl-Phenyl |
| 985 | Me | Me | OMe | OMe | O | 3-CF₃-4-Cl-Phenyl |
| 986 | Me | Et | OMe | OMe | O | 3-CF₃-4-Cl-Phenyl |
| 987 | Me | iPr | OMe | OMe | O | 3-CF₃-4-Cl-Phenyl |
| 988 | (CH₂)₄ | | OMe | OMe | O | 3-CF₃-4-Cl-Phenyl |
| 989 | (CH₂)₅ | | OMe | OMe | O | 3-CF₃-4-Cl-Phenyl |
| 990 | Me | Me | OMe | OMe | O | 3-CF₃-5-Cl-Phenyl |
| 991 | Me | Et | OMe | OMe | O | 3-CF₃-5-Cl-Phenyl |
| 992 | (CH₂)₄ | | OMe | OMe | O | 3-CF₃-5-Cl-Phenyl |
| 993 | (CH₂)₅ | | OMe | OMe | O | 3-CF₃-5-Cl-Phenyl |
| 994 | Me | Me | OMe | OMe | Bond | 3-CF₃-4-Cl-Phenyl |
| 995 | Me | Et | OMe | OMe | Bond | 3-CF₃-4-Cl-Phenyl |
| 996 | (CH₂)₄ | | OMe | OMe | Bond | 3-CF₃-4-Cl-Phenyl |
| 997 | (CH₂)₅ | | OMe | OMe | Bond | 3-CF₃-4-Cl-Phenyl |
| 998 | Me | Me | OMe | OMe | Bond | 4-Me-Phenyl |
| 999 | Me | Et | OMe | OMe | Bond | 4-Me-Phenyl |

| | | | | | |
|---|---|---|---|---|---|
| 1000 | (CH₂)₄ | OMe | OMe | Bond | 4-Me-Phenyl |
| 1001 | (CH₂)₅ | OMe | OMe | Bond | 4-Me-Phenyl |

Table 3 gives some of the compounds according to the invention given in table 1 in their salt form.

TABLE 3

| No. | Salt | R² | R³ | R⁵ | A | R⁶ |
|---|---|---|---|---|---|---|
| 34 | Trifluoromethane- | Me | Et | Me | O | 4-Cl-3-CF₃-Phenyl |
| | sulfonate | | | | | |
| 366 | Hydrochloride | Me | Et | Me | O | 4-F-3-CF₃-Phenyl |
| 371 | Trifluoroacetate | Me | Et | Me | O | 5-F-3-CF₃-Phenyl |
| 371 | Trifluoroacetate | Me | Et | Me | O | 5-F-3-CF₃-Phenyl |
| 373 | Trifluoroacetate | (CH₂)₅ | | Me | O | 5-F-3-CF₃-Phenyl |
| 373 | Hydrobromide | (CH₂)₅ | | Me | O | 5-F-3-CF₃-Phenyl |
| 373 | Trifluoroacetate | (CH₂)₅ | | Me | O | 5-F-3-CF₃-Phenyl |
| 538 | Acetate | Me | Et | Me | O | 2-Naphthyl |

For further characterization, log P data of some compounds are given in table 3. The log P data were determined in accordance with the EEC Directive 79/831 Annex V.A8 by HPLC (High Performance Liquid Chromatography) on a reversed-phase column (C18) using the following methods:

Temperature: 40° C.; Mobile Phase: 0.1% or 0.06% strength aqueous formic acid and 0.1% aqueous phosphoric acid and acetonitrile; linear gradient from 10% acetonitrile to 90% or 95% acetonitrile.

Calibration was carried out with the help of unbranched alkan-2-ones (consisting of 3 to 13 or 16 carbon atoms) with known logP values (determination of the logP values via the retention times by means of linear interpolation between two subsequent alkanones).

The lambda-max values were determined via the maxima of the chromatographic signals of the UV spectra from 190 nm to 400 or 450 nm.

TABLE 4

| | | | | | | ¹H-CDCl₃NMR |
|---|---|---|---|---|---|---|
| | | | | | | (300 MHZ), |
| | | | | | Melting point | chemical shift |
| No. | log p acidic | log p neutral | log p HCO₂H | log p H₃PO₄ | [° C.] | in ppm |
| 1 | 2.17 | 5.11 | | | | |
| 34 | 2.38 | | | | | |
| 36 | | 6.36 | 2.59 | | | |
| 130 | 2.05 | 4.53 | | | | |
| 226 | 1.68 | | | | | |
| 228 | 1.92 | 5.35 | 2.14 | | | |
| 250 | | | 2.45 | | | |
| 286 | | | 2.06 | | | |
| 290 | 2.51 | | | | | |
| 321 | 2.37 | 6.21 | | | | |
| 328 | 2.70 | 6.81 | | | | |
| 345 | | | 2.45 | | | |
| 356 | 2.93 | 7.01 | | | | |
| 365 | | | 2.37 | | | |
| 371 | 2.31 | 5.54 | | | | |
| 373 | 2.29 | | | | | |
| 373 (Trifluoroacetate) | 2.28 | | | | | |
| 373 (Hydrobromide) | 2.11 | | | | | |
| 388 | | 6.70 | 3.02 | | | |
| 393 | 2.24 | 5.62 | | | | |
| 398 | 2.27 | 5.59 | | | | |
| 401 | | 7.36 | | | | |
| 402 | | 6.60 | 2.96 | | | |
| 407 | 2.18 | | | | | |
| 440 | 2.96 | | 2.72 | | | |
| 445 | 2.71 | 5.74 | | | | |
| 446 | 2.69 | 6.30 | | | 91 | |
| 494 | | | | | | 1) |
| 498 | | | | | | 2) |
| 502 | 2.30 | | | | | |
| 547 | 1.74 | 4.33 | | | | |
| 555 | | | | 1.99 | | |
| 50 | 2.69 | 5.96 | | | | |
| 270 | | | 2.21 | | | |
| 538 | 2.38 | | | | | |
| 538 (Acetate) | 2.25 | | | | | |
| 559 | 2.76 | | | | | |
| 916 | 2.1 | 4.92 | | | | |
| 806 | | 5.91 | 2.79 | 2.74 | | |
| 986 | 2.23 | 4.14 | | | | |
| 987 | 2.35 | 4.5 | | | | |
| 999 | 1.89 | | | | | |
| 998 | 1.76 | | | | | |
| 836 | 2.71 | 5.74 | | | | |
| 928 | 2.3 | 5.31 | | | | |
| 892 | | 5.08 | 2.3 | 2.35 | | |
| 893 | | 5.49 | 2.44 | 2.51 | | |
| 894 | | | 2.55 | 1.73 | | |
| 902 | | 6.11 | 2.65 | 1.57 | | |
| 903 | | 6.54 | 2.8 | 2.98 | | |
| 904 | | 6.87 | 3.01 | 1.35 | | |
| 880 | | 5.44 | 2.47 | 2.63 | | |
| 888 | | 5.58 | 2.45 | 2.61 | | |
| 875 | | 5.51 | 2.38 | 2.56 | | |
| 870 | | 5.79 | 2.55 | 2.76 | | |
| 884 | | 5.86 | 2.69 | 2.87 | | |
| 889 | | 5.91 | 2.62 | 2.87 | | |
| 871 | | 5.84 | 2.54 | 2.79 | | |
| 882 | | 6.03 | 2.66 | 2.82 | | |
| 891 | | 6.21 | 2.64 | 2.82 | | |
| 878 | | 6.14 | 2.62 | 2.77 | | |
| 873 | | 6.42 | 2.79 | 3 | | |
| 886 | | 6.49 | 2.78 | 3.01 | | |
| 862 | 2.98 | | | | | |
| 906 | | 7.04 | 3.25 | 3.36 | | |
| 901 | | 6.72 | 3 | 3.12 | | |
| 899 | | 6.45 | 2.92 | 3.07 | | |
| 898 | | 6.12 | 2.78 | 2.88 | | |
| 816 | | 5.56 | 2.65 | 2.6 | | |
| 948 | | 5.74 | 2.98 | 2.87 | | |
| 846 | 2.79 | | | | | |
| 854 | 2.22 | | | | | |
| 949 | | 6 | 3.08 | 2.82 | | |
| 807 | | 6.13 | 2.82 | 2.7 | | |
| 951 | | 6.2 | 3.42 | 3.03 | | |
| 809 | | 6.41 | 3.04 | 2.81 | | |
| 830 | 2.4 | | | | | |
| 817 | | 5.88 | 2.8 | 2.74 | | |
| 819 | | 6.12 | 3 | 2.85 | | |
| 811 | | 6.64 | 3.17 | 3.16 | | |
| 944 | | 6.63 | 3.39 | 3.35 | | |
| 812 | | 6.81 | 3.31 | 3.35 | | |
| 814 | | 7.08 | 3.62 | 3.45 | | |
| 946 | | 7.1 | 3.68 | 3.61 | | |
| 804 | | 5.09 | 2.12 | 2.03 | | |
| 802 | | 4.84 | 2.08 | 2 | | |
| 801 | | 4.49 | 1.91 | 1.86 | | |
| 957 | | 4.71 | 1.97 | 2.07 | | |
| 958 | | 5.01 | 2.08 | 2.19 | | |
| 960 | | 5.26 | 2.22 | 2.29 | | |
| 825 | | 5.25 | 2.56 | 2.34 | | |
| 826 | | 5.61 | 2.78 | 2.53 | | |
| 815 | | 5.15 | 2.45 | 2.35 | | |
| 947 | | 5.39 | 2.78 | 2.61 | | |
| 810 | | 6.13 | 2.94 | 2.9 | | |
| 912 | | | 2.22 | | | |
| 761 | 2.94 | | | | | |
| 799 | 2.6 | | | | | |
| 608 | 2.01 | 5.31 | 2.01 | | 85.9 | |
| 615 | | | | | 99-101 | |
| 651 | | | | | 67-69 | |
| 721 | 2.21 | | | | | |
| 584 | | 6.54 | 2.84 | | | |
| 585 | | 6.34 | 2.89 | | | |
| 583 | | 6.09 | 2.78 | | | |
| 962 | 2.74 | 6.15 | | | | |
| 664 | | | | 2.13 | | |
| 663 | | | | 2.04 | 83 | |
| 665 | | | | 2.22 | | |
| 570 | | 6.37 | 2.95 | | | |
| 571 | | 6.79 | 3.03 | | | |
| 572 | | 6.70 | 2.87 | | | |
| 402 | | 6.60 | 2.96 | | | |
| 577 | | 6.94 | 3.24 | | | |
| 578 | 3.06 | | | | | |
| 589 | | 6.58 | 2.84 | | | |
| 591 | 3.21 | 6.92 | | | | |
| 590 | 2.93 | 7.01 | | | | |
| 981 | | 7.36 | 3.11 | | | |
| 587 | | 6.73 | 2.74 | | | |
| 568 | 2.86 | | | | | |
| 567 | 2.66 | | | | | |
| 566 | 2.83 | | | | | |
| 565 | 2.64 | | | | | |
| 564 | 2.51 | | | | | |
| 602 | 1.65 | 4.04 | | | | |
| 603 | 1.78 | 4.52 | | | | |
| 606 | 1.79 | 4.75 | | | | |
| 596 | 1.84 | 4.41 | 1.84 | | | |
| 597 | 1.90 | 5.09 | | | | |
| 600 | 1.90 | 5.34 | | | | |
| 573 | 2.90 | 7.14 | 2.90 | | | |
| 579 | 3.19 | 7.36 | 3.19 | | | |
| 697 | 2.67 | | | | | |
| 733 | 2.75 | | | | | |
| 669 | 2.38 | | | | | |
| 673 | 2.23 | | | | | |
| 656 | 2.37 | | | | | |
| 610 | 2.21 | | 2.21 | | | |
| 681 | 1.32 | | | | | |
| 612 | 1.72 | | | | | |
| 614 | 1.98 | | | | | |
| 620 | | | 2.20 | | | |
| 622 | | | 2.41 | | | |
| 681 | 1.32 | | | | | |

NMR data:
1) Re example No. 494: 2.5 (Me-butenyl radical). 3 (Me-amidine), 5.07-5.17 (m, CH₂-olefinic butenyl)
2) Re example No. 498: 1.19-1.28 (triplet CH₂ from OEt), 2.51 (Me-butenyl radical), 3 (Me-amidine), 4.94-5.0 (m, CH₂-olefinic butenyl).

### A. FORMULATION EXAMPLES

### 1. Dusting Agent

A dusting agent is obtained by mixing 10 parts by weight of a compound of formula (I) and 90 parts by weight of talc as inert substance and comminuting in a hammer mill.

### 2. Dispersible Powder

A wetable powder that is readily dispersible in water is obtained by mixing 25 parts by weight of a compound of formula (I), 64 parts by weight of kaolin-containing quartz as inert substance, 10 parts by weight of lignosulfonic potassium and 1 part by weight of oleoylmethyltauric sodium as wetting agent and dispersant and grinding in a pin mill.

### 3. Dispersion Concentrate

A dispersion concentrate that is readily dispersible in water is obtained by mixing 20 parts by weight of a compound of formula (I), 6 parts by weight of alkylphenol polyglycol ether (®Triton X 207), 3 parts by weight of isotridecanol polyglycol ether (8 EO) and 71 parts by weight of paraffinic mineral oil (boiling range e.g. ca. 255 to above 277° C.) and grinding to a fineness of below 5 microns in a friction ball mill.

### 4. Emulsifiable Concentrate

An emulsifiable concentrate is obtained from 15 parts by weight of a compound of formula (I), 75 parts by weight of cyclohexanone as solvent and 10 parts by weight of oxethylated nonylphenol as emulsifier.

### 5. Water-Dispersible Granules

Water-dispersible granules are obtained by mixing

```
75 parts by weight of a compound of formula (I),
10 parts by weight of calcium lignosulfonate,
  5 parts by weight of sodium lauryl sulfate,
  3 parts by weight of polyvinyl alcohol and
  7 parts by weight of kaolin,
```

grinding on a pin mill and granulating the powder in a fluidized bed by spraying on water as granulation liquid.

Water-dispersible granules are also obtained by homogenizing and precomminuting

```
25 parts by weight of a compound of formula (I),
  5 parts by weight of sodium 2.2′-dinaphthylmethane-6.6′-disulfonate,
  2 parts by weight of sodium oleoylmethyltaurate,
  1 parts by weight of polyvinyl alcohol,
17 parts by weight of calcium carbonate and
50 parts by weight of water
```

on a colloid mill, then grinding on a bead mill and atomizing and drying the suspension obtained in this way in a spray tower using a single-material nozzle.

### B. BIOLOGICAL EXAMPLES

### 1. Herbicidal Effect in the Preemergence Phase

Seeds of mono- and dicotyledonous broad-leaved weeds and crop plants are planted in wood-fiber pots in sandy loamy earth and covered with earth. The compounds of formula (I) formulated in the form of wettable powders (WP) or as emulsion concentrates (EC) are then applied to the surface of the covering earth as an aqueous suspension at a water application rate of converted 800 l/ha with the addition of 0.2% wetting agent.

Following treatment, the pots are placed in the greenhouse and kept under good growing conditions for the test plants. The visual assessment of the damage to the experimental plants was made after an experimental time of 3 weeks compared to untreated controls (herbicidal effect in percent (%): 100% effect=plants have died, 0% effect=as control plants). Here, the compounds at an application rate of 1.28 kg/ha for example exhibited in each case at least 80% effect against the harmful plants named in each case:

No. 114 against DIGSA, SETVI, CHEAL and VERPE,
No. 290 against DIGSA, AMARE, SETVI and VERPE,
No. 306 against SETVI, AMARE, MATCH and VERPE,
No. 345 against DIGSA, SETVI, CHEAL and MATCH,
No. 362 against DIGSA, SETVI and VERPE,
No. 364 against DIGSA, SETVI, ABUTH, AMARE, VERPE and VIOSS,
No. 478 against ECHCG, SETVI and VERPE,
No. 506 against ABUTH, VERPE and VIOSS,
No. 547 against DIGSA, ECHCG, SETVI, AMARE, CHEAL, GALAP and VIOSS,
No. 761 against SETVI, ABUTH, AMARE, VERPE, ECHCG,
No. 799 against SETVI, ABUTH, PHBPU, VERPE and VIOSS,
No. 893 against ECHCG, SETVI, AMARE, VERPE and VIOSS,
No. 894 against ECHCG, SETVI, AMARE, MATCH and VERPE,
No. 904 against SETVI, AMARE and VIOTR,
No. 962 against ECHCG, SETVI, ABUTH, AMARE, VERPE and VIOSS.

### 2. Herbicidal Effect in the Postemergence Phase

Seeds of mono- and dicotyledonous broad-leaved weeds and crop plants are planted in wood-fiber pots in sandy loamy soil, covered with earth and grown in the greenhouse under good growing conditions. 2 to 3 weeks after seeding, the experimental plants are treated at the one-leaf stage. The compounds of formula (I) formulated in the form of wettable powders (WP) or as emulsion concentrates (EC) are then sprayed onto the green plant parts in the form of an aqueous suspension at a water application rate of converted 800 l/ha with the addition of 0.2% wetting agent. After a standing time of the experimental plants in the greenhouse for ca. 3 weeks under optimal growing conditions, the effect of the preparation is assessed visually compared to untreated controls (herbicidal effect in percent (%): 100% effect=plants have died, 0% effect=as control plants). In this connection, for example the compounds at an application rate of 1.28 kg/ha exhibited in each case at least an 80% effect against the harmful plants specified in each case:

No. 36 against ABUTH, AMARE, CHEAL, PHBPU, VERPE and XANST,
No. 130 against ABUTH and VERPE,
No. 290 against ECHCG, ABUTH, AMARE, CHEAL, PHBPU, VERPE and SETVI,
No. 362 against ECHCG, ABUTH, AMARE, CHEAL, PHBPU and VERPE,
No. 364 against ECHCG, ABUTH, AMARE, CHEAL, PHBPU, VERPE and SETVI,
No. 365 against ECHCG, CHEAL, GALAP, PHBPU and POLSS,
No. 371 against ABUTH, GALAP, PHBPU and VERPE,
No. 401 against ECHCG, ABUTH, AMARE, VERPE, VIOSS and XANST,
No. 474 against ABUTH, AMARE, GALAP, PHBPU, VERPE and VIOSS,
No. 506 against VERPE and VIOSS,
No. 522 against VERPE and VIOSS,
No. 530 against AMARE, VERPE and VIOSS,
No. 761 against SETVI, ABUTH, AMARE, PHBPU, VERPE, VIOSS,
No. 799 against ABUTH, AMARE, PHBPU, VERPE and VIOSS,
No. 836 against AMARE, MATCH, PHBPU, VERPE, VIOSS and XANST,
No. 893 against SETVI, ABUTH, AMARE, VERPE and VIOSS,
No. 894 against SETVI, ABUTH, AMARE, MATCH, PHBPU and VIOSS,
No. 904 against ABUTH, AMARE, PHBPU and VIOSS,
No. 962 against ECHCG, ABUTH, AMARE, PHBPU, VERPE and VIOSS.

The abbreviations mean

| | | | |
|---|---|---|---|
| ABUTH | Abutilon theophrasti | AMARE | Amaranthus retroflexus |
| CHEAL | Chenopodium album | DIGSA | Digitaria sanguinalis |
| ECHCG | Echinochloa crus galli | GALAP | Galium aparine |
| MATCH | Matricaria | PHBPU | Pharbitis purpureum |
| | chamomilla | VERPE | Veronica persica |
| SETVI | Setaria viridis | XANST | Xanthium strumarium |
| VIOSS | Viola spec. | | |

1. A method for obtaining a herbicide comprising employing at least one compound of formula (I), and/or a salt thereof, 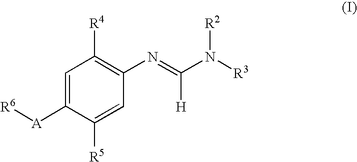
in which
R² and R³, independently of one another, are each (C₁-C₆)-alkyl, (C₃-C₆)-cycloalkyl, (C₂-C₆)-alkenyl, (C₂-C₆)-alkynyl, halo-(C₁-C₆)-alkyl, halo-(C₂-C₆)-alkenyl, halo-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl, or R² and R³ are together (CH₂)₄ or (CH₂)₅,
or
R² and R³ together with the nitrogen atom to which they are bonded, form a 5- or 6-membered saturated, partially saturated, unsaturated or aromatic ring which comprises k heteroatoms from the group consisting of oxygen, nitrogen and sulfur and which is substituted by p radicals from the group consisting of halogen, methyl, ethyl, methoxy, ethoxy, trifluoromethyl, nitro, cyano and hydroxy,
R⁴ and R⁵ independently of one another are each (C₁-C₆)-alkyl, (C₂-C₆)-alkenyl, (C₂-C₆)-alkynyl, (C₃-C₆)-cycloalkyl, halogen, cyano, hydroxy, mercapto, acyl, OR^{a}, SR^{a}, Si(R^{a})₃ halo-(C₁-C₆)-alkyl, (C₁-C₄)-alkoxy-(C₁-C₆)-alkyl or heterocyclyl bonded to phenyl via a carbon atom,
R^{a} is (C₁-C₈)-alkyl,
m is 1, 2 or 3,
R⁶ is in each case carbocyclyl or heterocyclyl substituted by n radicals from the group consisting of halogen, cyano, phenoxy, (C₁-C₈)-alkylcarbonyl, (C₁-C₈)-alkoxycarbonyl, (C₁-C₈)-alkyl, (C₁-C₈)-alkoxy, (C₂-C₈)-alkenyl, (C₂-C₈)-alkynyl and 1,3-dioxolan-2-yl, where the specified radicals (C₁-C₈)-alkyl, (C₁-C₈)-alkoxy, (C₂-C₈)-alkenyl and (C₂-C₈)-alkynyl are substituted by n radicals from the group consisting of (C₁-C₈)-alkoxy, hydroxy and halogen and where
1,3-dioxolan-2-yl is substituted by n radicals (C₁-C₈)-alkyl,
A is a bond or a divalent group —O—, —S(O)ₙ—, —NR⁹, —CR⁷═CR⁷—, —C≡C—, -A¹-, -A¹-A¹-, -A²-, -A³-, -A¹O—, -A¹S(O)ₙ—, —OA²-, —NR⁹-A²-, —OA²-A¹-, —OA²-CR⁷═CR⁸—, —S(O)-A¹-, —(CH₂)₂—ON═CR⁸—, —X-A²-NH—, —C(R⁸)═NO—(C₁-C₆)-alkyl or —O(A¹)ₖO—,
A¹ is in each case —CHR⁷—,
A² is in each case —C(═X)—,
A³ is —CR⁸═NO—,
X is in each case independently of the others oxygen or sulfur,
R⁷ is in each case independently of the others hydrogen, halogen, cyano, (C₁-C₆)-alkyl, (C₃-C₆)-cycloalkyl, phenyl, halogen, cyano, hydroxy, mercapto, halo-(C₁-C₆)-alkyl or (C₁-C₄)-alkoxy-(C₁-C₆)-alkyl,
R⁸ is in each case independently of the others hydrogen, (C₁-C₆)-alkyl, (C₂-C₆)-alkenyl, (C₂-C₆)-alkynyl, (C₁-C₆)-alkoxy, (C₁-C₆)-alkylthio, (C₃-C₆)-cycloalkyl, phenyl, halogen, cyano, hydroxy, mercapto, halo-(C₁-C₆)-alkyl, (C₁-C₄)-alkoxy-(C₁-C₆)-alkyl, carbocyclyl or heterocyclyl,
R⁹ is in each case independently of the others hydrogen, (C₁-C₆)-alkyl, carbocyclyl or heterocyclyl,
k is in each case independently of the others 1, 2 or 3,
n is in each case independently of the others n 0, 1 or 2, and
P is 0, 1, 2 or 3.

2. A method as claimed in claim 1, in which
R² and R³ independently of one another, are in each case (C₁-C₆)-alkyl, cyclopropyl, (C₂-C₆)-alkenyl, (C₂-C₆)-alkynyl, halo-(C₁-C₆)-alkyl, halo-(C₂-C₆)-alkenyl, halogen-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl or are together (CH₂)₄ or (CH₂)₅,
R⁴ is (C₁-C₆)-alkyl, halo-(C₁-C₆)-alkyl or (C₁-C₄)-alkoxy-(C₁-C₆)-alkyl,
R⁵ is halogen, (C₁-C₆)-alkyl, halo-(C₁-C₆)-alkyl or (C₁-C₄)-alkoxy-(C₁-C₆)-alkyl,
A is a bond, —O—, —S—, —CH₂CH₂—, —CH₂—, —OCH₂—, —CH═CH—, —C≡C—, —NH—CO—, —N(CH₃)—, NH— or —O—CO—NH—,
R⁶ is phenyl or naphthyl substituted by n radicals from the group consisting of halogen, cyano, phenoxy, (C₁-C₄)-alkylcarbonyl, (C₁-C₄)-alkyl, (C₁-C₆)-alkoxy, (C₂-C₆)-alkenyl, (C₂-C₆)-alkynyl and 1,3-dioxolan-2-yl, where the specified radicals (C₁-C₆)-alkyl, (C₁-C₆)-alkoxy, (C₂-C₆)-alkenyl and (C₂-C₆)-alkynyl are substituted by n radicals from the group consisting of (C₁-C₄)-alkoxy, hydroxy and halogen
and where 1,3-dioxolan-2-yl is substituted by n radicals (C₁-C₈)-alkyl,
or R⁶ is heterocyclyl substituted by n radicals from the group consisting of halogen, (C₁-C₆)-alkyl, halo-(C₁-C₄)-alkoxy and halo-(C₁-C₄)-alkyl,
m is 1 and
n is in each case independently of the others 0, 1 or 2.

3. The method as claimed in claim 1, in which
R² is methyl,
R³ is methyl, ethyl, cyclopropyl or isopropyl, or
R² and R³ are together (CH₂)₄ or (CH₂)₅,
R⁴ is methyl,
R⁵ is methyl or chlorine,
A is a bond, —O—, —S—, —CH₂—CH₂—, —CH₂—, —OCH₂— or —CH═CH—,
R⁶ is phenyl or naphthyl substituted by n radicals from the group consisting of halogen, cyano, phenoxy, (C₁-C₄)-alkylcarbonyl, (C₁-C₄)-alkyl, (C₁-C₆)-alkoxy, (C₂-C₆)-alkenyl, (C₂-C₆)-alkynyl and 1,3-dioxolan-2-yl, where the specified radicals (C₁-C₆)-alkyl, (C₁-C₆)-alkoxy, (C₂-C₆)-alkenyl and (C₂-C₆)-alkynyl are substituted by n radicals from the group consisting of (C₁-C₄)-alkoxy, hydroxy and halogen and where
1,3-dioxolan-2-yl is substituted by n radicals (C₁-C₈)-alkyl,
or R⁶ is pyridinyl, thiadiazolyl or thiazolyl substituted by n radicals from the group consisting of halogen, (C₁-C₆)-alkyl, halo-(C₁-C₄)-alkoxy and halo-(C₁-C₄)-alkyl,
m is 1 and
n is in each case independently of the others 0, 1 or 2.

4. A method for controlling undesired plants comprising employing at least one compound of formula (I), and/or a salt thereof 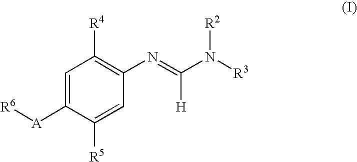
in which
R² and R³, independently of one another, are each (C₁-C₆)-alkyl, (C₃-C₆)-cycloalkyl, (C₂-C₆ alkenyl, (C₂-C₆)-alkenyl, halo-(C₁-C₆)-alkyl, halo-(C₂-C₆)-alkenyl, halo-(C₂-C₆)-alkenyl, (C₁-C₄)-alkoxy-(C₁-C₆)-alkyl, (C₁-C₄)-alkoxy-(C₇-C₆)-alkenyl or (C₁-C₄)-alkoxy-(C₂-C₆ or R² and R³ are together (CH₂)₄ or (CH₂)₅,
or
R² and R³ together with the nitrogen atom to which they are bonded, form a 5- or 6-membered saturated, partially saturated, unsaturated or aromatic ring which comprises k heteroatoms from the group consisting of oxygen, nitrogen and sulfur and which is substituted by p radicals from the group consisting of halogen, methyl, ethyl, methoxy, ethoxy, trifluoromethyl, nitro, cyano and hydroxy,
R⁴ and R⁵ independently of one another are each (C₁-C₆)-alkyl, (C₂-C₆)-alkenyl, (C₂-C₆)-alkynyl, (C₃-C₆)-cycloalkyl, halogen, cyano, hydroxy, mercapto, acyl, OR^{a}, SR^{a}, Si(R^{a})₃ halo-(C₁-C₄)-alkoxy-(C₁-C₆-alkyl or heterocyclyl bonded to phenyl via a carbon atom
R^{a} is (C₁-C₈)-alkyl,
m is 1, 2 or 3,
R⁶ is in each case carbocyclyl or heterocyclyl substituted by n radicals from the group consisting of halogen, cyano, phenoxy, (C₁-C₈)-alkylcarbonyl, (C₁-C₈)-alkoxycarbonyl, (C₁-C₈)-alkyl, (C₁-C₈)-alkoxy, (C₂-C₈)-alkenyl, (C₂-C₈)-alkynyl and 1,3-dioxolan-2-yl, where the specified radicals (C₁-C₈)-alkyl, (C₁-C₈)-alkoxy, (C₂-C₈)-alkenyl and (C₂-C₈)-alkynyl are substituted by n radicals from the group consisting of (C₁-C₈)-alkoxy, hydroxy and halogen and where
1,3-dioxolan-2-yl is substituted by n radicals (C₁-C₈)-alkyl,
A is a bond or a divalent group —O—, —S(O)ₙ—, —NR⁹, —CR⁷═CR⁷—, —C≡C—, -A¹-, -A¹-A¹-, -A²-, -A³-, -A¹O—, -A¹S(O)ₙ—, —OA²-, —NR⁹-A²-,
—OA²-A¹-, —OA²-CR⁷═CR⁸—, —S(O)ₙ-A¹-, —(CH₂)₂—ON═CR⁸—, —X-A²-NH—, —C(R⁸)═NO—(C₁-C₆)-alkyl or —O(A¹)ₖO—,
A¹ is in each case —CHR⁷—,
A² is in each case —C(═X)—,
A³ is —CR⁸═NO—,
X is in each case independently of the others oxygen or sulfur,
R⁷ is in each case independently of the others hydrogen, halogen, cyano, (C₁-C₆)-alkyl, (C₃-C₆)-cycloalkyl, phenyl, halogen, cyano, hydroxy, mercapto, halo-(C₁-C₆)-alkyl or (C₁-C₄)-alkoxy-(C₁-C₆)-alkyl,
R⁸ is in each case independently of the others hydrogen, (C₁-C₆)-alkyl, (C₂-C₆)-alkenyl, (C₂-C₆)-alkynyl, (C₁-C₆)-alkoxy, (C₁-C₆)-alkylthio, (C₃-C₆)-cycloalkyl, phenyl, halogen, cyano, hydroxy, mercapto, halo-(C₁-C₆)-alkyl, (C₁-C₄)-alkoxy-(C₁-C₆)-alkyl, carbocyclyl or heterocyclyl,
R⁹ is in each case independently of the others hydrogen, (C₁-C₆)-alkyl, carbocyclyl or heterocyclyl,
k is in each case independently of the others 1, 2 or 3,
n is in each case independently of the others n 0, 1 or 2, and
p is 0, 1, 2 or 3.

5. A method as claimed in claim 4, wherein the compound of formula (I) and/or salt is used for controlling undesired plants in crops of useful plants.

6. The method as claimed in claim 5, wherein the useful plants are transgenic useful plants.

7. A method of controlling undesired plants of claim 4 by using a compound and/or salt of formula (I) together with at least one formulation auxiliary.

8. A herbicidal composition comprising at least one compound of formula (I), and/or a salt thereof, and at least one formulation auxiliary 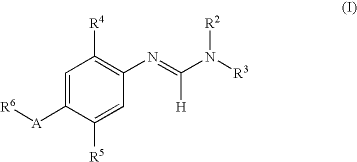
in which
R² and R³, independently of one another, are each (C₁-C₆)-alkyl, (C₃-C₆)-cycloalkyl, (C₂-C₆) alkenyl, (C₂-C₆)-alkynyl, halo-(C₁-C₆)-alkyl, halo-(C₂-C₆)-alkenyl, halo-(C₂-C₆)-alkynyl, (C₁-C₄)-alkoxy-(C₁-C₆)-alkyl, (C₁-C₄)-alkoxy-(C₇-C₆)-alkenyl or (C₁-C₄)-alkoxy-(C₂-C₆ or R² and R³ are together (CH₂)₄ or (CH₂)₅,
or
R² and R³ together with the nitrogen atom to which they are bonded, form a 5- or 6-membered saturated, partially saturated, unsaturated or aromatic ring which comprises k heteroatoms from the group consisting of oxygen, nitrogen and sulfur and which is substituted by p radicals from the group consisting of halogen, methyl, ethyl, methoxy, ethoxy, trifluoromethyl, nitro, cyano and hydroxy,
R⁴ and R⁵ independently of one another are each (C₁-C₆)-alkyl, (C₂-C₆)-alkenyl, (C₂-C₆)-alkynyl, (C₃-C₆)-cycloalkyl, halogen, cyano, hydroxy, mercapto, acyl, OR^{a}, SR^{a}, Si(R^{a})₃ halo-(C₁-C₄)-alkoxy-(C₁-C₆)-alkyl or heterocyclyl bonded to phenyl via a carbon atom,
R^{a} is (C₁-C₈)-alkyl,
m is 1, 2 or 3,
R⁶ is in each case carbocyclyl or heterocyclyl substituted by n radicals from the group consisting of halogen, cyano, phenoxy, (C₁-C₈)-alkylcarbonyl, (C₁-C₈)-alkoxycarbonyl, (C₁-C₈) alkyl, (C₁-C₈)-alkoxy, (C₂-C₈)-alkenyl, (C₂-C₈)-alkynyl and 1,3-dioxolan-2-yl,
where the specified radicals (C₁-C₈)-alkyl, (C₁-C₈)-alkoxy, (C₂-C₈)-alkenyl and (C₂-C₈)-alkynyl are substituted by n radicals from the group consisting of (C₁-C₈)-alkoxy, hydroxy and halogen and where
1,3-dioxolan-2-yl is substituted by n radicals (C₁-C₈)-alkyl,
A is a bond or a divalent group —O—, —S(O)ₙ—, —NR⁹, —CR⁷═CR⁷—, —C≡C—, -A¹-, -A¹-A¹-, -A²-, -A³-, -A¹O—, A¹S(O)ₙ—, —OA²-, —NR⁹-A²-,
—OA²-A¹-, —OA²-CR⁷═CR⁸—, —S(O)ₙ-A¹-, —(CH₂)₂—ON═CR⁸—, —X-A²-NH—, —C(R⁸)═NO—(C₁-C₆)-alkyl or —O(A¹)ₖO—,
A¹ is in each case —CHR⁷—,
A² is in each case —C(═X)—,
A³ is —CR⁸═NO—,
X is in each case independently of the others oxygen or sulfur,
R⁷ is in each case independently of the others hydrogen, halogen, cyano, (C₁-C₆)-alkyl, (C₃-C₆)-cycloalkyl, phenyl, halogen, cyano, hydroxy, mercapto, halo-(C₁-C₆)-alkyl or (C₁-C₄)-alkoxy-(C₁-C₆)-alkyl,
R⁸ is in each case independently of the others hydrogen, (C₁-C₆)-alkyl, (C₂-C₆)-alkenyl, (C₂-C₆)-alkynyl, (C₁-C₆)-alkoxy, (C₁-C₆)-alkylthio, (C₃-C₆)-cycloalkyl, phenyl, halogen, cyano, hydroxy, mercapto, halo-(C₁-C₆)-alkyl, (C₁-C₄)-alkoxy-(C₁-C₆)-alkyl, carbocyclyl or heterocyclyl,
R⁹ is in each case independently of the others hydrogen, (C₁-C₆)-alkyl, carbocyclyl or heterocyclyl,
k is in each case independently of the others 1, 2 or 3,
n is in each case independently of the others n 0, 1 or 2, and
p is 0, 1, 2 or 3.

9. A herbicidal composition of claim 8 further comprising at least one further herbicide active ingredients and/or optionally further formulation auxiliaries.

10. A composition of claim 8, wherein
R² and R³ independently of one another, are in each case (C₁-C₆)-alkyl, cyclopropyl, (C₂-C₆)-alkenyl, (C₂-C₆)-alkynyl, halo-(C₁-C₆)-alkyl, halo-(C₂-C₆)-alkenyl, halogen-(C₂-C₆)-alkynyl, (C₁-C₄)-alkoxy-(C₁-C₆)-alkyl, (C₁-C₄)-alkoxy-(C₂-C₆)-alkenyl or (C₁-C₄)-alkoxy-(C₂-C₆)-alkynyl or are together (CH₂)₄ or (CH₂)₅,
R⁴ is (C₁-C₆)-alkyl, halo-(C₁-C₆)-alkyl or (C₁-C₄)-alkoxy-(C₁-C₆)-alkyl,
R⁵ is halogen, (C₁-C₆)-alkyl, halo-(C₁-C₆)-alkyl or (C₁-C₄)-alkoxy-(C₁-C₆)-alkyl,
A is a bond, —O—, —S—, —CH₂CH₂—, —CH₂—, —OCH₂—, —CH═CH—, —C≡C—, —NH—CO—, —N(CH₃)—, NH— or —O—CO—NH—,
R⁶ is phenyl or naphthyl substituted by n radicals from the group consisting of halogen, cyano, phenoxy, (C₁-C₄)-alkylcarbonyl, (C₁-C₄)-alkyl, (C₁-C₆)-alkoxy, (C₂-C₆)-alkenyl, (C₂-C₆)-alkynyl and 1,3-dioxolan-2-yl, where the specified radicals (C₁-C₆)-alkyl, (C₁-C₆)-alkoxy, (C₂-C₆)-alkenyl and (C₂-C₆)-alkynyl are substituted by n radicals from the group consisting of (C₁-C₄)-alkoxy, hydroxy and halogen
and where 1,3-dioxolan-2-yl is substituted by n radicals (C₁-C₈)-alkyl,
or R⁶ is heterocyclyl substituted by n radicals from the group consisting of halogen, (C₁-C₆)-alkyl, halo-(C₁-C₄)-alkoxy and halo-(C₁-C₄)-alkyl,
m is 1 and
n is in each case independently of the others 0, 1 or 2.

11. A composition of claim 8, wherein
R² is methyl,
R³ is methyl, ethyl, cyclopropyl or isopropyl, or
R² and R³ are together (CH₂)₄ or (CH₂)₅,
R⁴ is methyl,
R⁵ is methyl or chlorine,
A is a bond, —O—, —S—, —CH₂—CH₂—, —CH₂—, —OCH₂— or —CH═CH—,
R⁶ is phenyl or naphthyl substituted by n radicals from the group consisting of halogen, cyano, phenoxy, (C₁-C₄)-alkylcarbonyl, (C₁-C₄)-alkyl, (C₁-C₆)-alkoxy, (C₂-C₆)-alkenyl, (C₂-C₆)-alkynyl and 1,3-dioxolan-2-yl, where the specified radicals (C₁-C₆)-alkyl, (C₁-C₆)-alkoxy, (C₂-C₆)-alkenyl and (C₂-C₆)-alkynyl are substituted by n radicals from the group consisting of (C₁-C₄)-alkoxy, hydroxy and halogen and where
1,3-dioxolan-2-yl is substituted by n radicals (C₁-C₈)-alkyl,
or R⁶ is pyridinyl, thiadiazolyl or thiazolyl substituted by n radicals from the group consisting of halogen, (C₁-C₆)-alkyl, halo-(C₁-C₄)-alkoxy and halo-(C₁-C₄)-alkyl,
m is 1 and
n is in each case independently of the others 0, 1 or 2.
